(19) United States
(12) Patent Application Publication
(10) Pub. No.: US 2023/0148895 A1
(43) Pub. Date: May 18, 2023

(54) SYSTEM AND METHOD FOR TRACKING MIGRATION OF A STRUCTURE

(71) Applicant: Aston University, Birmingham (GB)

(72) Inventors: Muhammad Moid Khalid KHAN, Birmingham (GB); Sarah JUNAID, Birmingham (GB); Laura LESLIE, Birmingham (GB); Subodh DESHMUKH, Birmingham (GB); Kanthan THEIVENDRAN, Birmingham (GB)

(21) Appl. No.: 17/995,634

(22) PCT Filed: Mar. 26, 2021
(86) PCT No.: PCT/EP2021/058055

§ 371 Date: Oct. 6, 2022

(30) Foreign Application Priority Data
Apr. 8, 2020 (GB) 2005220.5

(51) Int. Cl. A61B 5/06; A61B 5/00

(52) U.S. Cl. CPC A61B 5/062; A61B 5/4851; A61B 5/725; A61B 5/726; A61B 5/7203; A61B 5/458; A61B 5/6878; A61B 2562/0223; A61B 2562/046

(57) ABSTRACT

A system for tracking relative displacement between a first element and a second element in a structure, the system includes: a permanent magnet fixed relative to the first element; a magnetic sensor fixed relative to the second element, the magnetic sensor configured to measure a magnetic field strength; and a processor configured to determine the relative displacement between the first element and the second element, based on the magnetic field strength measured by the magnetic sensor. This invention has particular applications in e.g. orthopaedic prostheses. A related method includes the steps of measuring, using the magnetic sensor, a magnetic field strength; and determining the relative displacement between the relative displacement between the first element and the second element, based on the magnetic field strength measured by the magnetic sensor.

### TECHNICAL FIELD OF THE INVENTION

The present invention relates to a system and method for tracking the relative displacement between a first element and a second element, by using a permanent magnet and a magnetic sensor.

### BACKGROUND TO THE INVENTION

It is useful to measure displacement between two points in a structure, and in particular the change in that displacement over time. This can apply a vast range of physical structures, on a vast range of scales. For example, it can be useful to measure the amount by which two parts of a prosthetic joint move, relative to each other, over a given timescale. This invention was developed with a view to detection of aseptic loosening of an elbow joint, but it will be appreciated that the principles of the invention may be applied to various other orthopaedic prostheses, including knee and hip prostheses, as well as other (i.e. non-prosthetic, or non-anatomical) structures entirely.

Aseptic loosening of an elbow prosthesis is considered to be a key contributor in the failure of total elbow arthroplasty (i.e. surgery in which the elbow joint is totally replaced with a prosthesis). It has been found that relative migration of the implant from its original position leads to ingrowth of fibrous tissue, rather than bone. It is for this reason that proper fixation of the implant is essential, i.e. to avoid unnecessary growth of fibrous tissue. This can only be achieved if there is a stable interface between the implant and bone. According to various different studies, relative motion of more than 150 μm tends to give rise to undesirable growth of fibrous tissue. Bone ingrowth is generally favoured when the relative motion between the implant and the bone is below the 150 μm mark.

Several designs of elbow prostheses have ten-year survival rates ranging from 63% to 91%. In spite of these new designs, aseptic loosening remains a problem that affects the longevity of elbow prostheses. Compared with the survival rates of total knee and hip arthroplasty, which are considerably higher at 90% and 95% respectively, the survival rate of total elbow arthroplasty is 79.5%. It is clear to see that there is a long way to go in improving the longevity of elbow prostheses, particularly with regard to aseptic loosening.

Early detection of loosening is key to identifying early bone loss, and the “silent failure” often seen in total elbow arthroplasty. Early detection would reduce the burden on the clinical team, and in doing so would reduce the hospital costs, and allow clinicians and researchers learn about the loosening behaviour and therefore be able to design better implants to prevent such loosening. Our understanding of the causes and mechanism of aseptic loosening is still minimal, and this is due primarily to a lack of continuous monitoring. So, diagnosing early aseptic loosening remains a challenge.

Currently, x-ray imaging is used in clinical practice to detect loosening at regular time intervals (for example once or twice a year). However, early stages of loosening are ambiguous, and can be subjective due to low specificity and accuracy, and there is a high rate of detection of false positives. X-ray imaging is therefore limited in its ability to provide an accurate and reliable picture of loosening, but at present remains the gold standard technique. Other imaging modalities have also been carried out in detecting prosthesis loosening which includes: arthrography, bone scintigraphy, magnetic resonance imaging (MRI), and 18-fluorodeoxyglucose positron emission tomography (FCG-PET). The main issue with these techniques is that their respective detection performances are variable, they can time-consuming, it can be difficult to interpret the results, they can be observer-dependent, and costly. This makes these methods inappropriate for detecting early migration of a prosthesis.

At present, the high precision radiographic method for detecting aseptic loosening is Radiostereometric Analysis (RSA) technique. RSA can measure early signs of prosthesis displacement within the first postoperative year. However, this technique can only be adopted as a clinical research tool, and can only be used in a small number of patients.

The radiographic and RSA techniques are not suitable for prolonged monitoring, because they must be performed by a radiologist, and the prolonged exposure to radiation has obvious health risks.

Another technique that may be used is vibrometry, in which loosening is detected based on measuring the propagated vibrations of a tibia component of a knee prosthesis using 3D accelerometers. This technique, however, only has 20% higher sensitivity and specificity than the radiographic techniques, and at the moment has never been tested in respect of elbow prostheses. However in order to perform this technique, it is necessary to modify the implanted elbow prosthesis—which is not viable, as the design of the implants has been optimized through many years of research.

### SUMMARY OF THE INVENTION

The present invention aims to address the issues, identified above, with prior art techniques for improved detection of loosening of structures such as elbow prostheses, and in particular prolonged monitoring. Broadly speaking, the present invention provides a solution to this problem in the provision of a detection system in which the measurement of a magnetic field is used to detect the linear and angular displacement of an element. Specifically, a first aspect of the present invention provides a system for tracking relative displacement between a first element and a second element in a structure, the system including: a permanent magnet fixed relative to the first element; a magnetic sensor fixed relative to the second element, the magnetic sensor configured to measure a magnetic field strength; and a processor configured to determine the relative displacement between the first element and the second element, based on the magnetic field strength measured by the magnetic sensor.

This system presented in this application has many advantages which include low cost, accuracy, durability, measurement sensitivity and robustness to different material (liquid, non-magnetic material etc.).

It should be noted here that the term “relative displacement” should be interpreted broadly to cover both the scalar value of distance, and also the vector value of displacement, i.e. having both a magnitude (the distance) and a direction. The term “magnetic field strength” as used in this application may be used to refer to either of the following: magnetic field strength (usually denoted H or H, and alternatively referred to as “magnetic field intensity”, “magnetic field”, or “magnetizing field”), or magnetic flux density (usually denoted B or B, and alternatively referred to as “magnetic induction” or “magnetic field”), or any equivalent measurable physical effect which is proportional to either of these two. The skilled person is aware that in a vacuum the magnetic flux density and the magnetic field strength are the same, but that in a material (as will likely be the case in this invention) the magnetic field strength is given by:

$$H ≡ \frac{B}{{μ}_{0}} - M$$

Here, M is the magnetization of the material, which is the vector field which represents the density of permanent or induced magnetic dipole moments in a magnetic material.

In some cases, the magnetic sensor may be configured to generate magnetic field strength data based on the measured magnetic field strength, and to transmit this data to the processor. Then, the processor may be configured to convert this magnetic field strength data into relative displacement data, for example by using a predetermined algorithm (to be discussed in detail later). The system may further include a memory, and the processor may be configured to store the relative displacement data and optionally, the magnetic field strength data in the memory. Alternatively, in other cases, the magnetic sensor may be configured to transmit the magnetic field strength data to the memory, and the processor may then be configured to access this data in order to determine the relative displacement between the first element and the second element, thereby generating relative displacement data, which may then also be stored in the memory. In such cases, the system may be configured to delete the magnetic field strength data once the relative displacement data has been stored to the memory. The processor may be configured to generate the relative displacement data continuously. The system may include a sensing module which includes the magnetic sensor, and optionally, the processor or the memory. In configurations in which either the processor or memory is not located within with sensing module, the sensing module preferably includes a transmission means (preferably a wireless transmission means) for transmitting the magnetic field strength data and/or relative displacement data out of the sensing module, to whichever components which are located outside the sensor module.

As discussed, the invention is directed primarily to the tracking of the relative displacement between the first element and the second element, rather than taking a single measurement. In order to better achieve this, the magnetic sensor may be configured continuously to measure the magnetic field strength, thereby continuously generating magnetic field strength data. In other cases, the magnetic sensor may be configured to measure the magnetic field strength and thereby to generate the magnetic field strength data at predetermined intervals, and optionally to transmit the magnetic field strength data to the processor or memory at predetermined intervals. The frequency with which the measurement takes place (i.e. the inverse of the time interval between successive measurements) may be referred to herein as the “sampling frequency”, and may be between 1 and 10,000 Hz. The device may be configurable to utilize one of a plurality of frequency modes, the plurality of modes including at least a first mode and a second mode, and optionally a third mode. Any of these modes may be an ultra-low power mode with a sampling frequency of 1 to 100 Hz, preferably 5 to 50 Hz, and most preferably around 10 Hz. Throughout this application the term “around” should be interpreted to refer to a variation of ±10%. Another of the modes may be a low power mode, with a sampling frequency of 10 to 1,000 Hz, preferably 50 to 500 Hz, and most preferably around 100 Hz. Another of the modes may be a master controlled mode having a sampling frequency around 1,000 to 10,000 Hz, preferably 2,000 to 5,000 Hz, and most preferably around 3,300 Hz.

The device may be calibrated. In other words, the processor or a memory of the device may have stored thereon calibration data, wherein the relative displacement between the first element and the second element is determined based on both the magnetic field strength and the calibration data. The calibration data may include a predetermined magnetic field strength and a predetermined relative displacement between the first element and the second element, optionally in the form of magnetic field strength data, and predetermined relative displacement data. Specifically, the when the first element is separated from the second element by the predetermined relative displacement, the magnetic field strength measured at the magnetic sensor is the predetermined magnetic field strength. This effectively provides a baseline measurement from which other measurements may be calibrated. The processor may be configured to determine the difference between the measured magnetic field strength and the predetermined magnetic field strength, and to determine the relative displacement between the first element and the second element based on this difference, and the calibration data. The processor may be configured to determine the deviation between the predetermined relative displacement and the actual relative displacement between the first element and the second element, based on e.g. the deviation between the predetermined magnetic field strength and the measured magnetic field strength. If the predetermined relative displacement is known, and the deviation is known, then it is straightforward to calculate the actual relative displacement between the first element and the second element. In some cases, where all that is required is to track the change in the relative displacement between the first element and the second element (sometimes referred to herein as the “migration”) there is no need to calculate the actual (i.e. absolute) relative displacement between the two elements. A detailed explanation of the calibration process is given in the detailed description, later in this application.

The magnetic sensor is preferably configured to measure a vector value of the magnetic field strength, i.e. both a magnitude of the magnetic field strength, and its direction at a given location. Preferably, the magnetic sensor is configured to measure three orthogonal components of the magnetic field strength at the sensor, which are referred to herein as the x-component (or Bₓ), y-component (or B_{y}), and the z-component (or B_{z}). In this respect, the sensor itself may be considered to have three corresponding axes. For example, the magnetic sensor may include three one-dimensional, or scalar (e.g. Hall effect) sub-sensors arranged mutually orthogonally, each of the three one-dimensional sensors configured to measure a magnitude of a respective one of the three orthogonal components of the magnetic field at the sensor. The direction of the component of the magnetic field at the sensor of which a given sub-sensor is configured to measure or determine the magnitude is referred to the “orientation” of that sub-sensor. The orientations of the three sub-sensors are thus preferably mutually orthogonal. The orientations of the sub-sensors preferably correspond to the axes of the magnetic sensor, mentioned above.

Alternatively, the processor may be configured to convert, for example using a conversion module, magnetic field strength data including both the magnitude of the magnetic field and its direction into magnetic field strength data including the component of the magnetic field strength in each of three mutually orthogonal directions. The processor is preferably also able to convert between different coordinate systems, for example the processor may be configured to convert the components of the magnetic field in Cartesian coordinates into the components of the magnetic field in cylindrical, polar, or spherical coordinates.

The permanent magnet is preferably a cylindrical magnet, and it is preferred that the z-axis of the cylinder (i.e. the long or longitudinal axis) is parallel to the z-axis of the magnetic sensor, as defined earlier in this application. A cylindrical magnet is preferred in the present invention because it is able to produce a greater degree of magnetization as compared to other shaped magnets, such as rings, spheres, or tubes. It is particularly preferred that the z-axis of the cylinder is not only parallel, but also aligned with the z-axis of the sensor. In other words, the z-axis of the cylinder and the z-axis of the magnetic sensor are preferably collinear. On implantation, or at another time, the system is preferably calibrated by ensuring that the z-axis of the magnetic sensor is aligned with the z-axis of the magnetic field of the magnet. In other words, it is preferably that the z-axis of the cylindrical magnet is initially aligned with the z-axis of the sensor, and that the z-axis of the cylindrical magnet and the z-axis of the magnetic sensor are preferably initially collinear.

The operation of the system will now be described. Note that a detailed mathematical description of the determination of the relative displacement between the first element and the second element will be set out later in this application.

The magnetic sensor is configured to determine the value of the magnitude of the z-component of the magnetic field strength of the permanent magnet. In other words, the magnetic sensor is configured to determine the magnitude of B_{z} that would be measured by the sensor if the z-axes of the magnet and the sensor were aligned. Based on this value, the processor may be configured to determine the z-distance between the permanent magnet and the magnetic sensor. Herein, the term “z-distance” refers to the distance between two points in the z-direction. The processor may be configured to determine the z-distance based on a predetermined relationship between the z-component of the magnetic field strength of the permanent magnet and the z-distance. One such relationship is the following:

$${B}_{Z\_T} ( z ) = \frac{{μ}_{0} M}{2} ( \frac{z + {H}_{m}}{\sqrt{{( z + {H}_{m} )}^{2} + {( \frac{{D}_{m}}{2} )}^{2}}} - \frac{z}{\sqrt{{z}^{2} + {( \frac{{D}_{m}}{2} )}^{2}}} )$$

The terms in this relationship are defined later on in this application. It will be appreciated that this equation cannot be solved analytically for z. So, the processor may be configured to solve this equation numerically. For example, the processor may be configured to input a plurality of trial values of z into this equation, and to select the value of z as the value which results in a value of B_{z_T} which is closest to the value of the magnitude of the z-component of the magnetic field strength of the permanent magnet measured by the magnetic sensor.

With knowledge of the z-distance, and the x- and y-components of the magnetic field strength, it is then possible to calculate the values of the x-distance and the y-distance, and thus to calculate the relative displacement between the permanent magnet and the magnetic sensor. Specifically, the processor may be configured to determine the x-distance based on at least the z-distance, and the values of the magnitudes of the x- and z-components of the magnetic field strength at the magnetic sensor, measured by the magnetic sensor. Similarly, the processor may be configured to determine the y-distance based on at least the z-distance, and the values of the magnitudes of the y- and z-components of the magnetic field strength at the magnetic sensor, measured by the magnetic sensor. In order to do so, the processor may be configured to utilize the below relationships:

$$\begin{matrix}x = z ( \frac{{B}_{x}}{{B}_{z}} ) \\ y = z ( \frac{{B}_{y}}{{B}_{z}} )\end{matrix}$$

These equations are derived, and explained in more detail in the “detailed description” section of this application.

It will be appreciated that, in addition to varying as the z-distance changes, the value of the z-component of the magnetic field at the sensor varies as the x- and y-distances change. Similarly, the values of the x- and y-components of the magnetic field at the sensor vary as the z-distance changes. This is known as the cross-talk effect. With displacement in the z-direction, the value of the z-distance which is used to calculate the deviations in the x- and y-directions will become inaccurate. In order to address this, in some cases the present invention is configured to periodically update the value of the z-distance which it uses to calculate the x- and y-distances. It will be recalled that the magnetic sensor may either continuously generate magnetic field strength data, or to measure the magnetic field strength at predetermined intervals. The processor may be configured to update the z-distance value at regular intervals. For example, when the magnetic sensor is configured to continuously generate magnetic field strength data, the processor may be configured to update the z-distance value at regular intervals in time. Alternatively, or additionally, when the magnetic sensor is configured to measure the magnetic field strength at predetermined intervals, the processor may be configured to update the z-distance after a predetermined number of measurements of magnetic field strength have been taken. In other words, the processor may be configured to update the z-distance value after a predetermined number of samples have been taken.

We now discuss how the z-distance is updated. In some cases, the z-distance may simply be recalculated using the methods outlined previously in this disclosure. However, in other cases, after the predetermined amount of time, or after the predetermined number of measurements have been taken, the processor may be configured to recalculate the z-distance to generate a new z-distance, and then the processor may be configured to compare the new z-distance with the old z-distance. If the new z-distance differs from the old z-distance by more than or equal to a predetermined difference threshold, then the processor is configured to adopt the new z-distance. If the new z-distance differs from the old z-distance by less than the predetermined difference threshold, the processor is configured to reject the new z-distance, and to continue calculating the x-distance and y-distance using the old z-distance.

There are different types of magnetic sensors, which utilize various techniques and technologies to detect and measure the magnetic field strength. The various techniques or phenomena which may be employed to measure the magnetic field strength include, non-exhaustively, the following: Hall Effect, anisotropic magnetoresistance (AMR), giant magnetoresistance (GMR), Flux gate effect, induction coil effect, MEMS Lorentz force and other magnetic phenomena. Preferably the magnetic sensor is configured to measure the magnetic field strength at a resolution from 10⁻¹² to 10⁻³ Tesla (i.e. picoTesla to milliTesla). It is preferable to use a Hall Effect sensor to measure the magnetic field strength, because they are compact in size, low cost, and easy to integrate with data acquisition systems. As the skilled person is aware, Hall Effect sensors consist of a thin sheet of semiconductor known as Hall element. When a continuous current is flowing across the Hall element and there is no magnetic field, the current distribution is the same with no potential difference seen. However, when this sensor is subjected to any magnetic field, a Lorentz force deflects the charge carriers resulting in a potential difference. A Hall Effect sensor is generally able to measure a magnetic field strength of up to 1 Tesla. Generally, commercially available Hall Effect sensors are able to measure a magnetic field in three axes with very little power consumption. The magnetic sensor preferably also includes a temperature sensor for thermal drift compensation, and a digital output, optionally via an I2C or SPI bus, in order to integrate with the processor. Preferably, all of the components of the magnetic sensor are integrated on a single chip. The dimensions of the chip may, in some cases be equal to or less than 3 mm×3 mm×1 mm. Examples of magnetic sensors which may be used are: TLV493D/E (Infineon, Neubiberg, Germany), MLX90393 (Melexis, leper, Belgium) and AS54XX (Ams, Premstaetten, Austria).

In order to further improve the accuracy of the magnetic field strength readings taken by the magnetic sensor of the present invention, it is preferable that the system may include a plurality of sensors. For example, the system may include two magnetic sensors, or three magnetic sensors. However, in preferred configurations the system includes four magnetic sensors: a first magnetic sensor, a second magnetic sensor, a third magnetic sensor, and a fourth magnetic sensor. The system may include a sensing module or a sensing formation which includes the plurality of magnetic sensors. In configurations of the invention in which there are four magnetic sensors, the sensors may be arranged in a cross formation. The first sensor and the second sensor (herein, the “first pair of sensors”) may be separated in a first direction, and the third sensor and the fourth sensor (herein, the “second pair of sensors”) may be separated in a second direction, preferably perpendicular or substantially perpendicular to the first direction. The imaginary line connecting the first pair of sensors preferably bisects the imaginary line connecting the second pair of sensors. Preferably, each of the plurality of magnetic sensors is configured to measure the three orthogonal components of the magnetic field strength, as discussed in detail above with respect to the single magnetic sensor. Preferably, each of the plurality of magnetic sensors is identical to the other magnetic sensors, i.e. the system may include a plurality of identical magnetic sensors. The first direction is preferably parallel to the y-direction, and the second direction is preferably parallel to the x-direction. The plurality of magnetic sensors are preferably mounted with the same orientation. The plurality of magnetic sensors are preferably fixed in relation to each other, i.e. relative movement between the magnetic sensors is not possible.

In arrangements in which there are a plurality of magnetic sensors, it is preferable that the sensors are arranged in the x-y plane, and that the value of the z-component of the magnetic field strength at the plurality of sensors may be calculated by taking an average of the value of the z-component of the magnetic field strength as measured by each of the plurality of magnetic sensors. This ensures a more accurate measurement of the z-component of the magnetic field strength than is obtainable with a single sensor. The values of the x- and or y-components of the magnetic field strength at the plurality of sensors may be calculated in the same way. Alternatively, the values of the x- and y-components of the magnetic field strength may be calculated as a linear superposition of the values of the x- or y-components of the magnetic field strengths as measured by each sensor. More details about this are given later in this application, with reference to a specific implementation of the invention.

When data is received continuously, or quasi-continuously (i.e. at high frequency intervals) from the magnetic sensor, it may be susceptible to high frequency noise, which cannot be removed by a conventional finite impulse response (FIR) filter, which is a filter whose impulse response is of finite duration, because usually settles to zero in time, an example of which is a simple moving average filter. In preferred cases, the processor is configured to apply a Savitzky-Golay (SG) filter, or a modified SG filter (detailed description later on in this application) to the magnetic field strength data, or to the relative displacement data, in order to smooth it, and to remove the high frequency noise. A SG filter is basically a low pass filter, or a type of FIR filter which is also able to preserve the high frequency content of the desired signal. This in contrast to a simple FIR filter, in which the high frequency content of both the noise and the signal is removed.

Alternatively, the noise component of the signal may be removed using a discrete wavelet transform (DWT) denoising technique. DWT denoising techniques provide effective denoising with minimal computational complexity. Specifically, the processor may be configured to perform a DWT denoising technique on the magnetic field strength data, or to the relative displacement data (either may be referred to as the “data” in the remainder of this paragraph, for conciseness). The technique may include any or all of the following steps: transforming the data into the wavelet domain, optionally by selecting a mother wavelet function from a wavelet family, for example the Sym 6 wavelet. The processor may be configured to define a decomposition level, for example 6, which was found to smooth out the signal well with minimal reduction to the actual signal information. The processor may be configured then to reduce selected components of the coefficients of the wavelet transform, optionally by selection of a thresholding function. The preferred thresholding function is the Stein's Unbiased Risk Estimate (SURE) threshold. A mathematical definition of this thresholding function is set out later in this disclosure. The processor may then be configured to select the thresholding selection rule. In the present case, the preferred thresholding selection rule is global thresholding in which the noise is assumed to have Gaussian distribution having the same amplitude and frequency distribution that span the same data length. Finally, the reduced coefficients may be rescaled and inversely transformed to give the denoised signal.

The disclosure above relates to the method and the processing used by the system of the present invention, but now we turn to the structural elements. As discussed at the outset of this application, an important application of the invention is for determining migration of prosthetic joints. Specifically, the system may be used to detect aseptic loosening, which arises from a failure of the bond between the implant and the bone, leading to migration of the implant. The term “aseptic” here indicates that the loosening occurs because of the failure of the bond over time, rather than due to infection. However, it is noted that the present invention could equally well be used to detect migration of an implant due to infection. Accordingly, in some cases, the first element may include a prosthesis, preferably an orthopaedic prosthesis such as an artificial elbow joint. An important purpose of the invention is to track the migration of the prosthesis relative to the bone. So, in such cases, the second element preferably includes a component which is fixable to a bone. Accordingly, the magnet may be fixed relative to the bone. Alternatively, the magnet itself may be fixable to the bone.

It is important that orthopaedic prostheses are secured tightly to bone. This is generally achieved by filling the space between the prosthesis and the bone into which it is implanted with bone cement. In such cases, the system may further include a cement restrictor which is configured to prevent the cement from diffusing further into the bone undesirably. When loosening of the prosthesis occurs, the prosthesis becomes detached from the bone cement, and is thus able to migrate within a person's body. Overall, therefore the system may include: a first element comprising an orthopaedic prosthesis, and a second element which is fixable to a bone, and optionally bone cement filling the space between the orthopaedic prosthesis and the bone, and optionally a cement restrictor. The magnetic sensor is preferably attached to or embedded into the prosthesis. The magnet is preferably fixable to the bone, fixed to the cement restrictor, or embedded within the bone cement. In this way, when the prosthesis becomes displaced from the bone cement, the relative displacement between the magnet and the magnetic sensor changes, thereby causing the magnetic field strength measured by the sensor to change, which change can be converted into a change in the relative displacement between the two. It should be noted that in the above arrangement, the magnet and magnetic sensor could be switched around, and the same technical effect would be achieved. A detailed description of the structural features of the invention with reference to the drawings will be given later in this application.

A second aspect of the invention provides a method of tracking relative displacement between a first element and a second element in a structure, the structure including a magnet fixed to the first element and a magnetic sensor fixed to the second element, and the method including the steps of: measuring, using the magnetic sensor, a magnetic field strength; and determining the relative displacement between the relative displacement between the first element and the second element, based on the magnetic field strength measured by the magnetic sensor. It will be appreciated that the second aspect of the invention may be performed by systems according to the first aspect of the invention. The optional features set out above with respect to the first aspect of the invention apply equally well here, where compatible. Certain optional features of the invention are set out below, though this does not constitute an exhaustive list.

In some cases, the method may include the step of the magnetic sensor generating magnetic field strength data based on the measured magnetic field strength, and transmitting this data to a processor. The method may include a step of converting this magnetic field strength data into relative displacement data, for example by using a predetermined algorithm (to be discussed in detail later). This step may be performed by the processor. The method may further include a step of storing the relative displacement data and optionally, the magnetic field strength data, for example in a memory. Alternatively, in other cases, the magnetic sensor may transmit the magnetic field strength data to a memory, and the method may further include a step of accessing this data in order to determine the relative displacement between the first element and the second element, thereby generating relative displacement data, which may then also be stored in a memory. In such cases, the method may include a step of deleting the magnetic field strength data once the relative displacement data has been stored to the memory. The step of generating relative displacement data may include generating the relative displacement data continuously.

As discussed, the invention is directed primarily to the tracking of the relative displacement between the first element and the second element, rather than taking a single measurement. In order to better achieve this, the step of generating magnetic field strength data may include continuously generating magnetic field strength data. In other cases, the step of generating magnetic field strength data may include measuring the magnetic field strength at predetermined intervals, and optionally transmitting the magnetic field strength data to e.g. a processor or memory at predetermined intervals. The frequency with which the measurement takes place (i.e. the inverse of the time interval between successive measurements) may be between 1 and 10,000 Hz. The device may be configurable to utilize one of a plurality of frequency modes, the plurality of modes including at least a first mode and a second mode, and optionally a third mode. Any of these modes may be an ultra-low power mode with a sampling frequency of 1 to 100 Hz, preferably 5 to 50 Hz, and most preferably around 10 Hz. Throughout this application the term “around” should be interpreted to refer to a variation of ±10%. Another of the modes may be a low power mode, with a sampling frequency of 10 to 1,000 Hz, preferably 50 to 500 Hz, and most preferably around 100 Hz. Another of the modes may be a master controlled mode having a sampling frequency around 1,000 to 10,000 Hz, preferably 2,000 to 5,000 Hz, and most preferably around 3,300 Hz.

The device may be calibrated. In other words, the processor or a memory of the device may have stored thereon calibration data, wherein the “determining step” may include determining the relative displacement between the first element and the second element based on both the magnetic field strength and the calibration data. The calibration data may include a predetermined magnetic field strength and a predetermined relative displacement between the first element and the second element, optionally in the form of magnetic field strength data, and predetermined relative displacement data. Specifically, the when the first element is separated from the second element by the predetermined relative displacement, the magnetic field strength measured at the magnetic sensor is the predetermined magnetic field strength. This effectively provides a baseline measurement from which other measurements may be calibrated. The method may include a step of determining the difference between the measured magnetic field strength and the predetermined magnetic field strength, and determining the relative displacement between the first element and the second element based on this difference, and the calibration data. The method may include a step of determining the deviation between the predetermined relative displacement and the actual relative displacement between the first element and the second element, based on e.g. the deviation between the predetermined magnetic field strength and the measured magnetic field strength. If the predetermined relative displacement is known, and the deviation is known, then it is straightforward to calculate the actual relative displacement between the first element and the second element. In some cases, where all that is required is to track the change in the relative displacement between the first element and the second element (sometimes referred to herein as the “migration”) there is no need to calculate the actual (i.e. absolute) relative displacement between the two elements. A detailed explanation of the calibration process is given in the detailed description, later in this application.

The step of measuring preferably includes measuring a vector value of the magnetic field strength, i.e. both a magnitude of the magnetic field strength, and its direction at a given location. Specifically, this step preferably includes measuring three orthogonal components of the magnetic field strength at the sensor, which are referred to herein as the x-component (or Bx), y-component (or By), and the z-component (or Bz). In this respect, the sensor itself may be considered to have three corresponding axes as described in more detail with respect to the first aspect of the invention.

The method may further include converting, for example using a conversion module, magnetic field strength data including both the magnitude of the magnetic field and its direction into magnetic field strength data including the component of the magnetic field strength in each of three mutually orthogonal directions. The method may also include converting between different coordinate systems, where necessary, for example converting the components of the magnetic field in Cartesian coordinates into the components of the magnetic field in cylindrical, polar, or spherical coordinates.

The permanent magnet is preferably a cylindrical magnet, and it is preferred that the z-axis of the cylinder (i.e. the long or longitudinal axis) is parallel to the z-axis of the magnetic sensor, as defined earlier in this application. It is particularly preferred that the z-axis of the cylinder is not only parallel, but also aligned with the z-axis of the sensor. On implantation, or at another time, the system is preferably calibrated by ensuring that the z-axis of the magnetic sensor is aligned with the z-axis of the magnetic field of the magnet. In other words, the z-axis of the cylindrical magnet is preferably initially aligned with the z-axis of the sensor.

The operation of the system will now be described.

The magnetic sensor preferably determines the value of the magnitude of the z-component of the magnetic field strength of the permanent magnet. In other words, the magnetic sensor is configured to determine the magnitude of B_{z} that would be measured by the sensor if the z-axes of the magnet and the sensor were aligned. The method may then include a step of determining the z-distance between the permanent magnet and the magnetic sensor, based on the value of the magnitude of the z-component of the magnetic field strength of the permanent magnet. The z-distance may be determined based on a predetermined relationship between the z-component of the magnetic field strength of the permanent magnet and the z-distance. One such relationship is the following:

$${B}_{Z\_T} ( z ) = \frac{{μ}_{0} M}{2} ( \frac{z + {H}_{m}}{\sqrt{{( z + {H}_{m} )}^{2} + {( \frac{{D}_{m}}{2} )}^{2}}} - \frac{z}{\sqrt{{z}^{2} + {( \frac{{D}_{m}}{2} )}^{2}}} )$$

The terms in this relationship are defined later on in this application. It will be appreciated that this equation cannot be solved analytically for z. The method may include a step of solving this equation numerically. For example, the method may include the steps of inputting a plurality of trial values of z into this equation, and selecting the value of z as the value which results in a value of B_{z_T} which is closest to the value of the magnitude of the z-component of the magnetic field strength of the magnet measured by the magnetic sensor.

The method may then include the step of determining the x-distance based on at least the z-distance, and the values of the magnitudes of the x- and z-components of the magnetic field strength at the magnetic sensor, measured by the magnetic sensor. The method may then also include the step of determining the y-distance based on at least the z-distance, and the values of the magnitudes of the y- and z-components of the magnetic field strength at the magnetic sensor, measured by the magnetic sensor. This may be done using the relationships set out earlier in this application.

The method may further include a step of periodically updating the value of the z-distance which it uses to calculate the x- and y-distances. The z-distance value may be updated at regular intervals. For example, when the magnetic sensor is configured to continuously generate magnetic field strength data, the method may include a step of updating the z-distance value at regular intervals in time. Alternatively, or additionally, when the magnetic sensor is configured to measure the magnetic field strength at predetermined intervals, the method may include a step of updating the z-distance after a predetermined number of measurements of magnetic field strength have been taken. The z-distance value may be updated after a predetermined number of samples have been taken.

We now discuss how the z-distance is updated, in the context of the second aspect of the invention. In some cases, the z-distance may simply be recalculated using the methods outlined previously in this disclosure. However, in other cases, after the predetermined amount of time, or after the predetermined number of measurements have been taken, the method may include the step of recalculating the z-distance to generate a new z-distance, and comparing the new z-distance with the old z-distance. If the new z-distance differs from the old z-distance by more than or equal to a predetermined difference threshold, the new z-distance may be adopted. If the new z-distance differs from the old z-distance by less than the predetermined difference threshold, new z-distance may be rejected, and to the method may include the step of continuing calculating the x-distance and y-distance using the old z-distance.

According to the second aspect of the invention, the magnetic sensor(s) may be as in the first aspect of the invention so that disclosure will not be repeated here.

In order to further improve the accuracy of the magnetic field strength readings taken by the magnetic sensor of the present invention, it is preferable that the system may include a plurality of sensors. The plurality of sensors may be as set out earlier in this disclosure.

In arrangements in which there are a plurality of magnetic sensors, the value of the z-component of the magnetic field strength at the plurality of sensors may be calculated by taking an average of the value of the z-component of the magnetic field strength as measured by each of the plurality of magnetic sensors. This ensures a more accurate measurement of the z-component of the magnetic field strength than is obtainable with a single sensor. The values of the x- and or y-components of the magnetic field strength at the plurality of sensors may be calculated in the same way. Alternatively, the values of the x- and y-components of the magnetic field strength may be calculated as a linear superposition of the values of the x- or y-components of the magnetic field strengths as measured by each sensor. More details about this are given later in this application, with reference to a specific implementation of the invention.

In preferred cases, the method may include the step of applying a Savitzky-Golay (SG) filter, or a modified SG filter (detailed description later on in this application) to the magnetic field strength data, or to the relative displacement data, in order to smooth it, and to remove the high frequency noise.

Alternatively, the noise component of the signal may be removed using a discrete wavelet transform (DWT) denoising technique. Specifically, the method may include the step of performing a DWT denoising technique on the magnetic field strength data, or to the relative displacement data (either may be referred to as the “data” in the remainder of this paragraph, for conciseness). The method may include any or all of the following steps: transforming the data into the wavelet domain, optionally by selecting a mother wavelet function from a wavelet family (for example Sym 6, as discussed above). The method may further include a step of defining a decomposition level (for example 6, as discussed above). The method may then include a step of reducing selected components of the coefficients of the wavelet transform, and optionally of selecting a thresholding function. The preferred thresholding function is the Stein's Unbiased Risk Estimate (SURE) threshold. A mathematical definition of this thresholding function is set out later in this disclosure. The method may further include a step of selecting a thresholding selection rule. In the present case, the preferred thresholding selection rule is global thresholding in which the noise is assumed to have Gaussian distribution having the same amplitude and frequency distribution that span the same data length. Finally, the reduced coefficients may be rescaled and inversely transformed to give the denoised signal.

### BRIEF DESCRIPTION OF THE DRAWINGS

Embodiments of the present invention, and experimental results will now be described with reference to the drawings, in which:

FIG. 1 shows a schematic diagram of a system for tracking migration of an elbow prosthesis.

FIG. 2 shows a cylindrical magnet and a Hardinge cement restrictor which may be used in the system of FIG. 1.

FIG. 3 shows a diagram of a cylindrical magnet illustrating its dimensions.

FIG. 4 shows the magnetic field vector distribution in the z-r plane around the cylindrical magnet.

FIG. 5 shows a plane contour plot of the radial component of the magnetic field strength (Bᵣ) around a cylindrical magnet.

FIG. 6 shows a plane contour plot of the z-component of the magnetic field strength (B_{z}) around a cylindrical magnet.

FIG. 7 shows a block diagram of the sensor open drain configuration.

FIG. 8 is a flowchart showing the algorithm which is used to determine the z-distance.

FIG. 9 is a set of diagrams illustrating movement in the x- and y-directions, along with the associated angles.

FIG. 10A shows the arrangement of four sensors S1 to S4 which are used in the “quad sesor configuration” described in the “experimental results” section below.

FIG. 10B shows a process by which a 2-bit address can be configured and allocated to each of sensors S1 to S4.

FIG. 11 is a flowchart illustrating a DWT denoising process.

FIG. 12 is a schematic diagram of the setup used to test the system of the present invention.

FIG. 13 is a plot of estimated vs. actual distance measured using a single sensor configuration.

FIG. 14 is a plot of displacement in the x-, y-, and z-directions over time for both filtered and unfiltered signals.

FIG. 15 is a plot demonstrating angular movement around the sensor.

FIG. 16 is a plot demonstrating the reduction in noise and cross-talk achieved by the present invention, for movement in the y-direction.

FIG. 17 is a plot demonstrating the reduction in noise and cross-talk achieved by the present invention, for movement in the z-direction.

FIG. 18 is a plot demonstrating the response of the system to movement in the y-direction with different materials located between the sensor and the magnet.

FIG. 19 includes four plots demonstrating the response of the system to movement in the y-direction at different, fixed z-distances.

FIG. 20 demonstrates the sensitivity of the system at different z-values.

FIG. 21 demonstrates the ability of the system to measure linear and angular displacement simultaneously.

FIG. 22 shows the response to movement in the y-direction of all four sensors in the quad sensor configuration.

FIG. 23 demonstrates the reduction in noise in the output of the quad sensors configuration as compared to the single sensor configuration.

FIG. 24 is a plot of estimated vs. actual z-distance for the quad sensor configuration.

FIG. 25 shows plots of quasi-static linear movement of the magnet in the y-direction, at constant x- and z-distances, showing both filtered and unfiltered plots.

FIG. 26 shows a comparison of linear movement detection between a single sensor configuration and a quad sensor configuration.

FIG. 27 shows filtered plots of quasi-static linear movement of the magnet in the x-direction, at constant y- and z-distances.

FIG. 28 shows a comparison of angular displacement detection between single sensor and quad sensor configuration.

FIG. 29 shows a comparison of raw magnetic data and calibrated quad sensor data for movement in the z-direction at constant y-distance, to demonstrate the improvements achieved by the present invention in terms of noise-reduction and cross-talk.

FIG. 30 shows a comparison of raw magnetic data and calibrated quad sensor data for movement in the y-direction at constant z-distance, to demonstrate the improvements achieved by the present invention in terms of noise-reduction and cross-talk.

FIG. 31 shows dynamic and quasi-static movement of the magnet in the y-direction at different z-distances.

FIG. 32 shows a comparison of the single sensor configuration with the quad sensor configuration at different z-values.

### DETAILED DESCRIPTION OF THE DRAWINGS

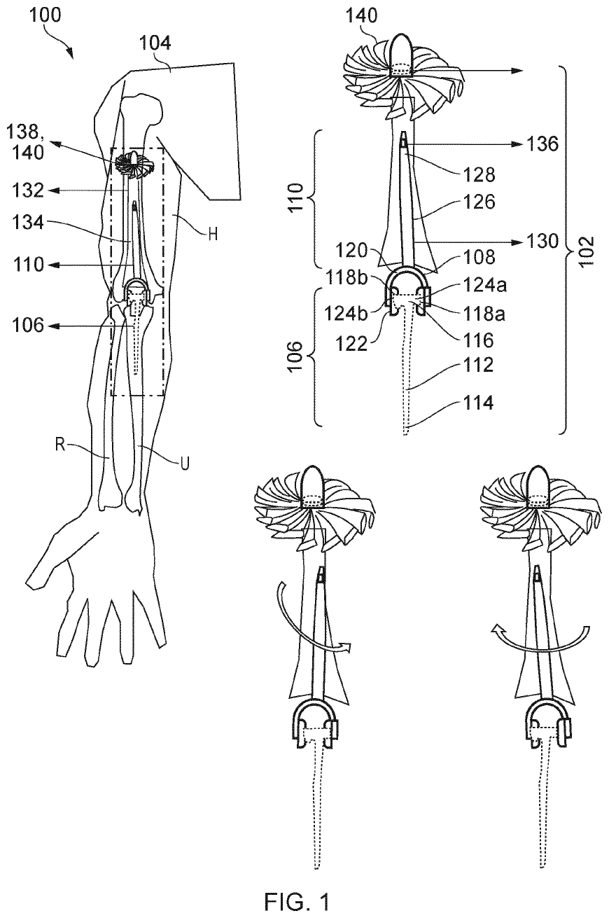

FIG. 1 shows an example of a system 100 of the present invention in use with a prosthetic elbow joint 102. The drawing shows a user's arm 104, and shows the humerus H, radius R, and ulna U bones. The elbow E is the joint between the humerus H on one side, and the radius R and ulna U on the other side. In FIG. 1, the user has had a prosthetic elbow joint 102 inserted. The prosthetic elbow joint 102 includes an ulnar component 106, a pivot 108, and a humeral component 110. The ulnar component 106 includes an elongate ulnar shaft 112, having a distal end 114, and a proximal end 116. The proximal end 116 is broad, and includes two lateral projections 118a, 118b. The pivot 108 includes a humeral pivot component 120 and an ulnar pivot component 122. The humeral pivot component 120 is rotatable relative to the ulnar pivot component 122. Specifically, the humeral pivot component 120 is rotatable relative to the ulnar pivot component 122 in a manner which mimics the rotation of a human elbow joint, i.e. it operates as a simple hinge joint, which is not able to hyperextend, and with very limited rotation. The ulnar pivot component 122 includes a pair of recesses 124a, 124b, each of which receives a respective lateral projection 118a, 118b of the proximal end 116 of the ulnar shaft 112. Preferably the lateral projections of 118a, 118b are not able to rotate within the recesses 124a, 124b, i.e. there is a tight or snug fit between the lateral projections 118a, 118b and their respective recesses 124a, 124b. The humeral component 110 preferably includes a humeral shaft 126 having a distal end 128 and a proximal end 130, the proximal end 130 being connected to the humeral pivot component 120. The humeral component 110 extends into a cavity 132 inside the humerus H. This cavity 132 is filled with bone cement 134. Preferably, the various components of the elbow prosthesis, specifically the ulnar component 106, the pivot 108 and the humeral component 110 are made of a cobalt-chromium or titanium alloy, since these alloys will have a negligible effect of the magnetic field, and the magnetic sensor 136 will be able to detect the magnetic field without any attenuation.

The bone cement 134 (either in the configuration shown in FIG. 1, or in any other conceivable configuration) is preferably an inert material which is able to withstand forces to which the human elbow joint is usually subject. It is preferably a polymer, and specifically it is preferably polymethylmethacrylate (PMMA). However, there are many types of bone cement that vary by their viscosity and antibiotic content. In total elbow arthroplasty the low viscosity cement is commonly used with or without antibiotics depending upon the surgeon's preference. PALACOS (Heraeus Noblelight Ltd, UK) low viscosity (PMMA) bone cement with and without antibiotic (Gentamicin) was used in the specific example which was used to obtain the results set out later in this application. The bone cement comes in two components made up of a powder (copolymer) and liquid (monomer). The two components are preferably mixed together at a ratio of 2:1 in a bowl in a fume cupboard (Airone FC 750 model, Safelab systems Ltd. Somerset, UK) for 2 to 3 minutes to form PMMA cement. While in a semi-solid form, the cement was poured into a pre-made mould to make PMMA slabs with thicknesses of 2.5 mm, 5 mm and 7.5 mm. The PMMA cement was left in the mould for 5 minutes to completely polymerize and harden before removing from the mould.

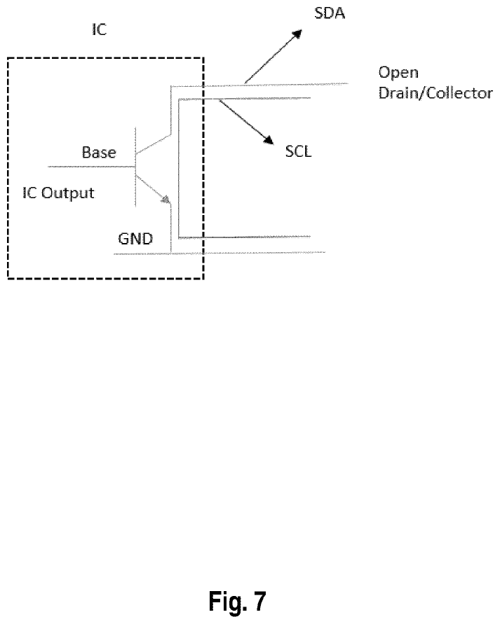

At the distal end 128 of the humeral shaft 126 is a magnetic sensor 136. The magnetic sensor 136 may take any of the forms suggested earlier in this application, as well as any other of which the skilled person may be aware. In preferred implementations, the magnetic sensor 136 is a magnetoresistive sensor. In the example which was used to obtain the results set out later in this application, the Infineon TLV493D magnetic sensor was used to detect magnetic field intensity in 3 orthogonal directions and from this, the prosthesis position can be determined. The PCB was designed for the sensor and then the sensor was enclosed in 2 mm thick titanium alloy (Ti-6AL-4V). Before calibrating the sensor according to the appropriate working envelope, the sensor 136 must be configured with the data acquisition device, since the sensor 136 is a digital sensor utilizing 120 communication protocol. NI MyRio was used as a data acquisition device to retrieve data from the magnetic sensor via its serial data pin (SDA) serial clock pin (SCL). As the magnetic sensor has the output via 120 protocol, two pull up resistors were required on the 120 line (SDA and SCL) as shown in FIG. 7. These resistors are necessary because the device has an open collector configuration. In open collector configuration, the system can only connect to the clock line (SCL) or signal data line (SDA) to ground but it cannot drive the lines to high. For the line to be able to go to the high voltage the pull up resistor must be inserted because we need a stable voltage state to define the two-binary state of bits i.e. 0 V as 0 bit and 3.3V as 1 bit. The value of the pull up resistor is important for the design configuration because an incorrect value of the resistor can lead to signal loss. By using the following equations the values of the pull up resistor can be calculated:

$${R}_{min} = \frac{{V}_{c c} - {V}_{OL ( Max )}}{{I}_{O L}}$$

In which Rₘᵢₙ is the minimum pull-up resistor value, V_{cc} is the supply voltage, V_{CL(Max)} and I_{CL} are low level output voltage and current respectively.

$${R}_{max} = \frac{{t}_{r}}{0.8473 * {C}_{b}}$$

In which Rₘₐₓ is the maximum pull-up resistor value, tᵣ is the rise time, and C_{b} is the bus capacitance. The pull-up resistor value can then be selected as any value between Rₘᵢₙ and Rₘₐₓ.

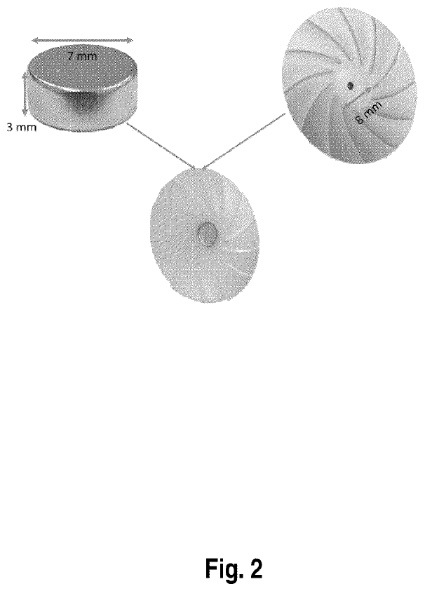

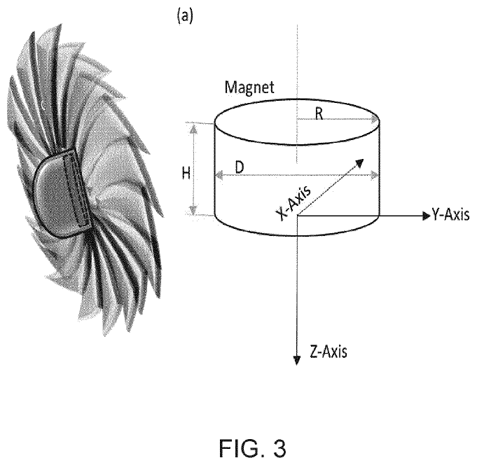

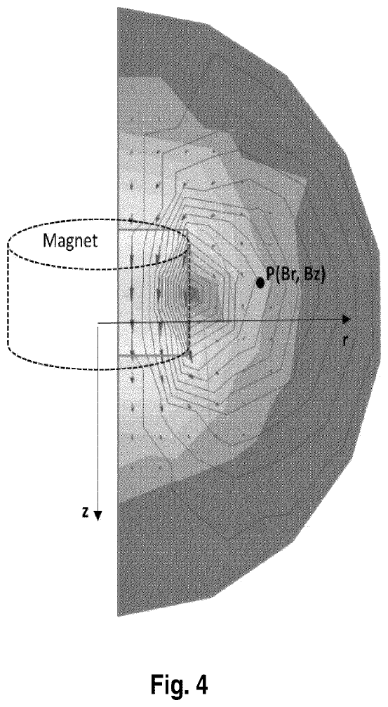

At the end of the matrix of bone cement 134 which is furthest from the pivot component 108, there is a permanent magnet 138 and a cement restrictor 140. In the present invention, the cement restrictor 140 is in the form of a disc of material including a number of slits cut into it, which is placed in the cavity 132 at its distal end, in order to prevent cement migration towards the shoulder S. The particular cement restrictor which was used to obtain the experimental results below was a Hardinge Cement Restrictor (see FIG. 2, which shows the cement restrictor 140 and the magnet 138). As discussed, in order to locate the position of the magnet, it is important to know its magnetic field strength. In the setup which was used to obtain the results set out below, an axis-magnetized cylindrical magnet was selected as a suitable source. A permanent magnet such as the example used here is a reliable source of steady magnetic field. The range and accuracy of magnetic field localization depends upon the magnetic remanence (which is the residual magnetization left behind in a ferromagnetic material after an external magnetic field is removed). The higher the magnetic remanence, the higher the range and accuracy. The remanence of the magnet depends on the size of the magnet: the bigger the magnet, the larger the magnetic remanence. In preferred cases, including the setup which was used to obtain the results below, a rare earth neodymium magnet (NdFeB, preferably Nd₂Fe₁₄B) was used, as it provides the highest available remanence to size ratio. An example of the magnet 138, is shown in FIGS. 2 and 3, in which the magnet 138 has a radius of 7 mm, and a height of 3 mm. In order to investigate the magnetic field of the permanent magnet 138, the inventors carried out an axisymmetric finite element model. FIG. 4 shows the magnetic field vector distribution in the z-r plane (because the simulation is axisymmetric, the magnetic field is axisymmetric around the z-axis of the magnet, and so the x-y plane can be considered as the r-plane). The magnetic field strength at any point P, and its specific coordinate point can be obtained by using the following equations:

$$\frac{{B}_{x}}{{B}_{y}} = \frac{x}{y} \\ {B}_{r} = \sqrt{{B}_{x}^{2} + {B}_{y}^{2}} \\ r = \sqrt{{x}^{2} + {y}^{2}}$$

Where Bₓ and B_{y} are the components of the magnetic field at point P in the x- and y-directions respectively, and x and y are the coordinate positions of point P on the x- and y-axes. Bᵣ is the magnetic field in the component of the magnetic field in the radial direction (i.e. the component of the magnetic field in the x-y plane), and r is the distance from the z-axis of the magnet in a direction parallel to the x-y plane.

Thus, the coordinate of the magnetic field at point P can be calculated by using the following equation.

$$x = r \frac{{B}_{x}}{{B}_{r}} \\ y = r \frac{{B}_{y}}{{B}_{r}}$$

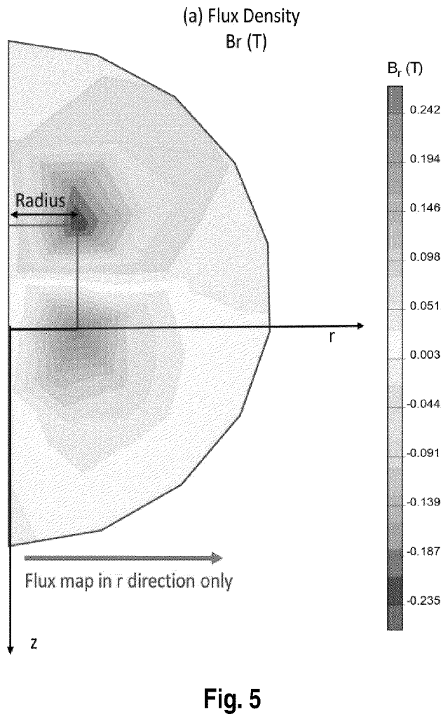

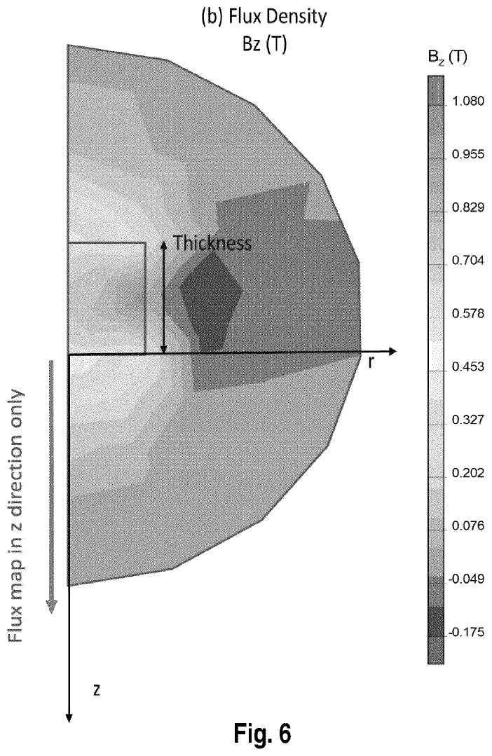

So, to obtain the three-axis displacement of the sensor from the three-axis magnetic field the relationship between (Bᵣ, Bᵣ) and (z, r) is enough where B_{z} is the magnetic field along the z axis of the sensor. FIG. 4 shows the contour plot of the magnetic field density of the permanent magnet (Bᵣ and B_{z}) in the z-r plane. FIGS. 5 and 6 indicate that the value of Br increases when r is increased, it reaches its maximum value when r=R, the radius of the magnet then Br decreases as r is increased and eventually goes to zero at longer distances. Also FIG. 5 indicates that the value of B_{z} decreases when z is increased and at distance z>16 mm the value of B_{z} is zero. To avoid multiple results when determining r and z from the magnetic field the movement of the magnet in r plane should preferably not exceed the specified regions as shown in plots and all the movement should be restricted between these regions. FIG. 6 shows a plot of the radial and z-direction components of the magnetic field of a permanent cylindrical magnet in the z-r plane. From this plot, the following features are apparent:
Nonlinearity: both B_{z} and Bᵣ in the z-r plane vary nonlinearly when the observation point moves away from the magnet.
Crosstalk effect: the value of Bᵣ varies according to the value of z as well as the value of r, and the value of B_{z} varies according to the value of r, as well as the value of z.

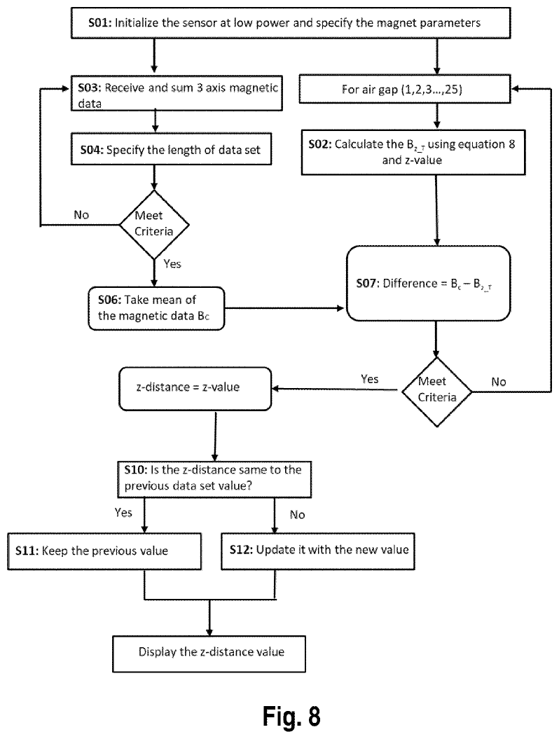

For these reasons, and as discussed, it is also necessary to calibrate the magnetic sensor 136. For example, to determine the distance between the magnet and sensor in one direction requires that we understand the interdependency of the output data with movement in the other axes. So, in this section a preferred method is described for the calibration of the magnetic sensor 136 which can be used to determine the change in relative displacement of the second element (i.e. the magnet 138) and the first element (i.e. the magnetic sensor 136). As the magnetic sensor has the capability of measuring magnetic field along three orthogonal axes simultaneously, the sensor can be used to measure field direction in two different planes. By using this concept, we can calibrate the sensor and identify its starting position. For the axis magnetised cylindrical permanent magnet the magnetic field along its axis (i.e. at a radius of zero) can be derived from:

$${B}_{Z \_ T} ( z ) = \frac{{μ}_{0} M}{2} ( \frac{z + {H}_{m}}{\sqrt{{( z + {H}_{m} )}^{2} + {( \frac{{D}_{m}}{2} )}^{2}}} - \frac{z}{\sqrt{{z}^{2} + {( \frac{{D}_{m}}{2} )}^{2}}} ) \qquad Eqn . 8$$

In which M is the axial magnetization of the magnetic, μ₀ is the relative magnetic permeability, and z is the distance from the pole of the magnet in the z-direction. Dₘ is the diameter of the magnet. The theoretical localization of the permanent magnet can be found by using the equation:

$$B = {B}_{x} + {B}_{y} + {B}_{z} = \frac{{μ}_{0} M}{2} ( \frac{z + {H}_{m}}{\sqrt{{( z + {H}_{m} )}^{2} + {( \frac{{D}_{m}}{2} )}^{2}}} - \frac{z}{\sqrt{{z}^{2} + {( \frac{{D}_{m}}{2} )}^{2}}} ) \qquad Eqn . 9$$

Where Bₓ, B_{y}, B_{z} are the three components of the magnetic flux density measured by the magnetic sensor 136 along its x-, y- and z-axes respectively. The overall method which is used to determine the z-distance from the measured magnetic field values, the algorithm shown in FIG. 8 may be used.

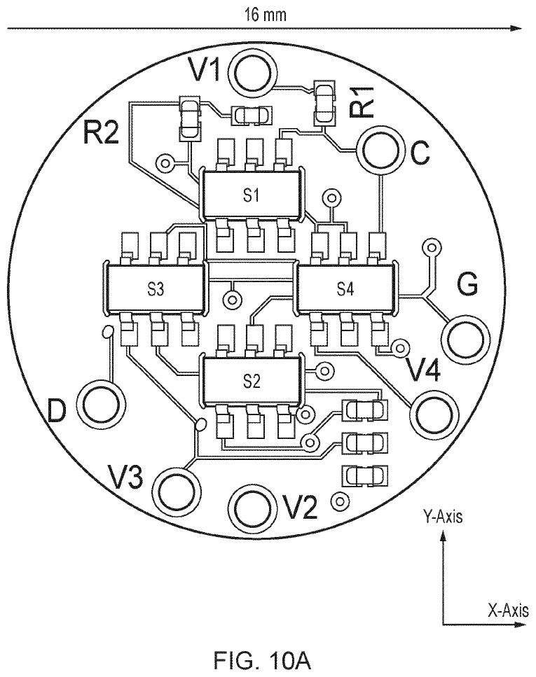

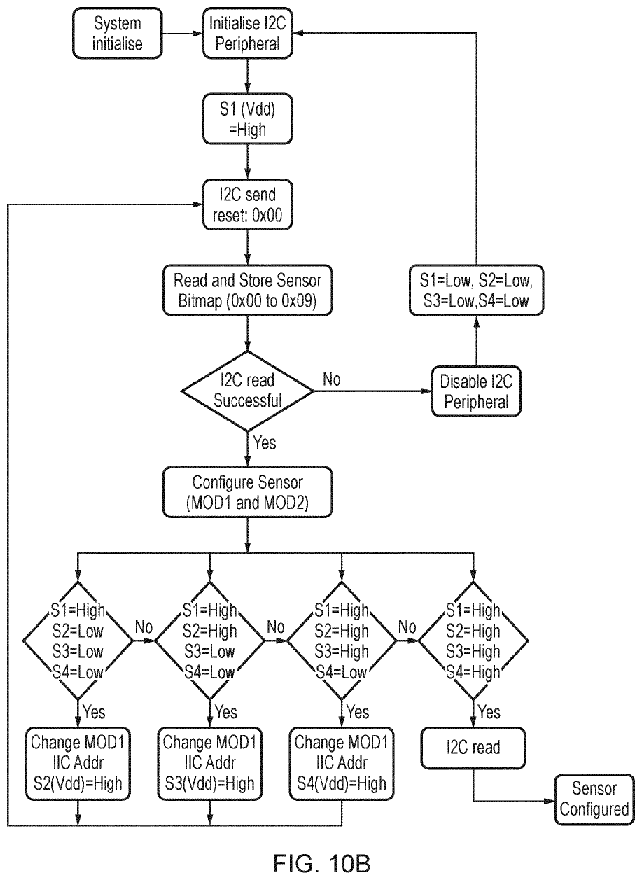

In some configurations, a plurality of magnetic sensors may be used, as illustrated in FIG. 10A. In this case, the calculations are slightly different. Here, in order to measure the radiograph-free migration of the elbow prosthesis, the inventors used a magnetic measuring system consisting of 2×2 magnetic sensing array (“quad sensor”) hermetically sealed in titanium metal, a permanent magnet embedded in the cement restrictor and a layer of PMMA cement between them to mimic the original implant position. The selection of the magnetic system was based on the human body transparency towards the magnetic field and the negligible effect of prosthesis material on magnetic flow. In this configuration, a compact 3D magnetic sensor (Infineon TLV493D) with digital output via the 120 bus was used to detect the position of the implant via measuring the magnetic field intensity emitted from the magnet in 3 orthogonal directions. The sensors were mounted on a rigid base (PCB) with a distance of 6 mm between sensors S1 and S2 in the y-direction and 6 mm between sensors S3 and S4 in the x-direction as shown in the FIG. 10A. The size of the rigid base (PCB) was designed based on the dimensions of a commercially available humeral stem diameter. According to Zimmer humeral stem, the diameter of the humeral stem varies from 6 mm to 20 mm. As the quad sensor configuration requires an area of 10 mm, an extra 6 mm area was added in the final PCB design for the auxiliary components. National Instrument (My DAQ RIO 1900) was used as a data acquisition device for configuring and retrieving data from the quad sensor. In the quad sensor configuration, each sensor needs to be configured via the 120 bus and should be allocated with a specific 2-bit address. FIG. 10B describes the configuration process for how to achieve the specific bit address for sensor S1, S2, S3, and S4. In configurations employing four sensors arranged as shown in FIG. 10A, the following equations may be used to calculate the values of Bₓ, B_{y}, and B_{z}:

$$Bxc = Bx 1 - Bx 2 + Bx 3 - Bx 4 \\ Byc = By 1 - By 2 + By 3 - By 4 \\ Bzc = \frac{{\sum }_{i = 1}^{n} {B}_{z} i}{n}$$

Thereafter, the same relations as outlined above apply. In order to determine the z-value, the method shown in FIG. 8 may be used. In a first step S01, the magnetic sensor 136 is initialized, and the magnet parameters are specified. In the present case, the magnet parameters may include (but are not limited to) the height of the magnet, the radius or diameter of the magnet, the axial magnetization of the magnet, the material from which the magnet is made. The following additional parameters may also be specified: the material properties (e.g. relative magnetic permeability) of the bone cement, or whichever material is located between the magnetic sensor and the magnet. Then in step S02, a theoretical magnetic field B_{z_T} is calculated or determined using equation 8 above, using a first z-value. In parallel with this calculation, in step S03, a three-axis magnetic field strength reading is taken from the magnetic sensor, and the three components of the magnetic field strength at the magnetic sensor are summed together. Then, in step S04, a length of the data set is specified. At step S05, it is determined whether the number of readings from the magnetic sensor is equal to the specified data set. If so, the method proceeds to step S06, in which an average of the sum of the magnetic field strength values is taken to give an average B₀. If not, then the method returns to step S03, and the process is repeated until the required number of readings has been taken.

In step S07, the values of B_{C} and B_{z_T} are compared, and in step S08 it is determined whether the difference between the two meets a certain criterion. If so, the z-value used to calculate B_{z_T} is deemed to be the “correct” value for the z-distance. In other words, if B_{C} and B_{z_T} differ by less than or equal to a predetermined threshold, the z-value which gives that value of B_{z_T} is determined to be the “correct” z-value in step S09, and the corresponding value of z, is noted. If the difference between the two exceeds the threshold, then the process returns to step S02, taking the next value of z, until a suitable value of B_{z_T} is arrived at. In some cases, the successive z-values which are input into the equations to determine B_{T} are at 0.01 mm intervals, but other intervals are also envisaged (e.g. 0.05 mm, 0.1 mm, 0.5 mm).

As discussed, the focus of the present invention is to track the change in relative displacement between the magnet and magnetic sensor. So, in step S10, it is determined whether the value of z is the same as the previous value. In preferred cases, the requirement is not that the z-value is exactly the same; rather, if the “new” z-value differs from the previous z-value by an amount which is less than a predetermined difference threshold, then the previous z-value is maintained, in step S11. And, if not, in step S11, the value of z is updated. Then, after a fixed interval (i.e. the sampling interval) the process is repeated. Now that the z distance is determined, it is possible to determine the linear displacements in the x- and y-axes as follows.

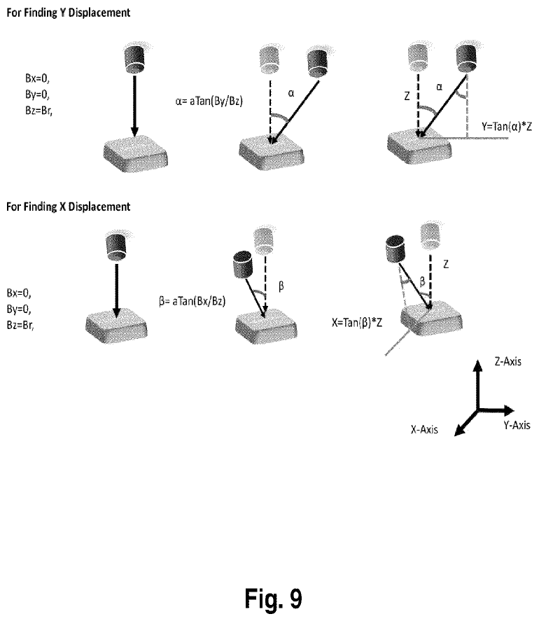

As shown in FIG. 9, when the magnet is displaced in the y-direction (with the z-distance remaining constant), it makes an angle of α with the z-axis, in the y-z plane. Similarly, when the magnet is displaced in the x-direction (with the z-distance remaining constant), it makes an angle of β with the z-axis in the x-z plane. The values of α and β may be found as follows:

$$α = {tan}^{- 1} ( \frac{{B}_{y}}{{B}_{z}} ) \\ β = {tan}^{- 1} ( \frac{{B}_{x}}{{B}_{z}} )$$

Then, the x- and y-distances may be found as follows:

x=z·tan β

y=z·tan α

Filtering the Results (i): Savitzky-Golay Filter

As discussed earlier in this application, the results can be improved by filtering the received data. The data received from the sensor need to be smoothened as it contain high frequency content that cannot be removed by plain FIR average filter. To achieve the high degree of noise removal from the desired signal, the length (N) of the signal has to be larger so that the signal bandwidth become greater than the filter pass band frequency.

$${ω}_{c} = \frac{π}{N}$$

In the current study, Savitzky-Golay (SG) filter also known as least-square or polynomial smoothing filter is used to smooth the desired signal. The SG filter is basically a low pass filter or it can be also consider as a type of finite impulse response FIR digital filter that can preserve the high-frequency content of the desired signal. The output signal from the sensor can be represented as:

y(n)=x(n)+w(n)

Where x(n) represents the magnetic field signal with high frequency content while w(n) is the associated noise with magneto resistive sensor i.e. Johnson (thermal noise), shot noise, 1/f (flicker) noise. The SG filter can be defined by two parameters that are denoted as K for the polynomial degree and M for sequence. The following assumptions are made in the SG filter:

I. All data points of the signal should be natural numbers.

II. The length of the signal should be N=2M+1 and is odd for the sequence of M.

III. Data points should be positioned symmetrically about the origin xₒ as follow:

x_{N}=[x_{-M}, . . . ,x₋₁,x₀,x₁, . . . ,x_{M}]

Polynomial smoothing of N samples of data is equivalent to replacing them by the values that lie on smooth polynomial curves drawn between the noisy samples.

\hat{x}ₘ=c₀+c₁m+ . . . +cₖm^{k},−M≤m≤M

Where \hat{x}ₘ represent the m^{th} smooth data point. The coefficients cᵢ are determined optimally by least square fit that minimize the least square error and also fits the given data on corresponding polynomial curve. For N data samples the performance index can be minimized as follows:

$$E {\sum }_{m = - k}^{k} {e}_{m}^{2} = {\sum }_{m = - k}^{k} ( {{x}_{m -} ( {c}_{0} + {c}_{1} m + … + {c}_{k} {m}^{k} )}^{2}$$

Similarly, we define K+1 polynomial basis vectors as follows:

S=[s₀,s₁, . . . ,sₖ]

Hence, the smooth data can be represented in vector form as:

$$\hat{x} = {\sum }_{i = 0}^{k} {c}_{i} {s}_{i} = {B}_{x}$$

As the data points should be symmetrically about the origin, the middle smoothed value y₀=\hat{x}₀ is given in terms of middle SG filter b₀.

$${y}_{0} = {b}_{0}^{T} x = {\sum }_{m = - M}^{M} {b}_{0} ( m ) {x}_{m}$$

The N-dimensional vector x can be shifted to n instants of time as follows:

x→[x_{n-M}, . . . ,xₙ₋₁,xₙ,xₙ₊₁, . . . ,x_{n+M}]

The resulting length N, order K, SG filter for smoothing a noisy sequence x(n) will be, in its steady-state form as:

$$y ( n ) = {\sum }_{m = - M}^{M} {b}_{0} ( - m ) x ( n - m )$$

Filtering the Results (ii): DWT Denoising.

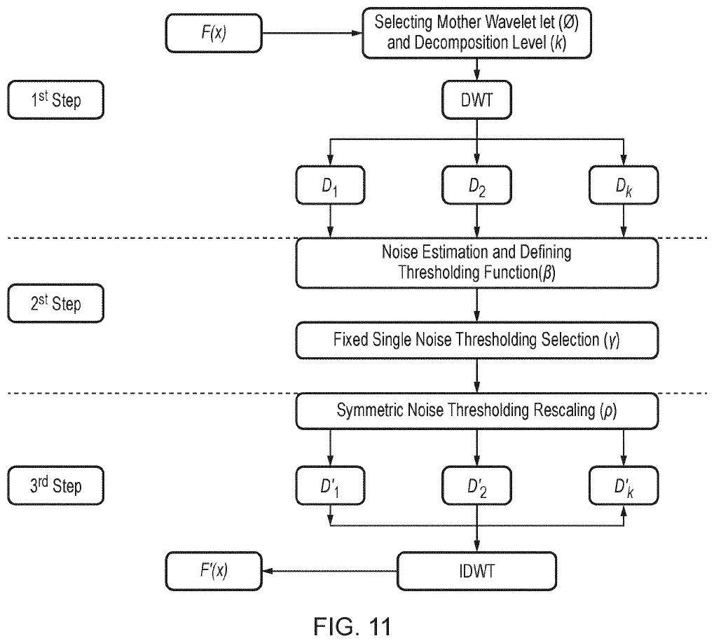

As discussed, the data received from the sensors can be difficult to analyse because it may contain high content of noise. Therefore the data may need to be smooth. In an alternative to Savitzky-Golay filtering, discrete wavelet transform (DWT) denoising techniques may be used to estimate the signal from the sensor and remove noise component. DWT provides an effective denoising with minimal computational complexity. The DWT denoising technique consists of three main steps with five parameters as shown in the flowchart in FIG. 11.

First, is the data from the sensor needs to be transformed into a wavelet domain with the length of the signal power of 2. This transformation can be done by selecting a mother wavelet function (ø) from the wavelet family. Selecting a suitable type of wavelet is extremely important for denoising the data because something two similar wavelets may give different denoising data. After selection of the wavelet function, the decomposition level (k) needs to be defined. Then, a criterion is selected to reduce or shrink the coefficient of the wavelet transform. The coefficient of the wavelet transform can be reduced by selecting a specific thresholding function (β). There are four types of thresholding functions that are commonly used. Among them, Stein's Unbiased Risk Estimate (SURE) threshold is mostly used because of its state of the art decomposition of noise and better performance. The SURE thresholding can be define as:

$$SURE ( t , x ) = N - 2 \times {M}_{( i : \vert {x}_{i} \vert \leq t )} + {\sum }_{i = 1}^{N} {( \vert {x}_{i} \vert ∧ t )}^{2}$$

Where x, is the detailed wavelet coefficient, t is the candidate threshold, N is the length of data and M is the number of the data points less than t. SURE thresholding is generally used to obtain the unbiased variance between the unfiltered and filtered data. After defining the function, the thresholding selection rule (γ) is selected. In DWT denoising, denoising of the signal depends greatly on the selection of the noise threshold. The wrong choice can result in lowering down the signal strength. Traditional thresholding transforms the coefficient whose magnitude is below the specified value. In the DWT denoising technique, there are different thresholding selection parameters but the most commonly used threshold is the global thresholding. In global thresholding noise is assumed to have Gaussian distribution, having the same amplitude and frequency distribution that span the same data length. Global thresholding can be further divided into soft and hard thresholding which can be defined respectively in the equations below:

$${x}_{j , i}^{′} = \{ \begin{matrix}{x}_{j , i} & : & \vert {x}_{j , i} \vert \geq t \\ 0 & : & \vert {x}_{j , i} \vert < t\end{matrix} \} \\ {x}_{j , i}^{′} = \{ \begin{matrix}sign ( {x}_{j , i} ) ( \vert {x}_{j , i} \vert - t ) & : & \vert {x}_{j , i} \vert \geq t \\ 0 & : & \vert {x}_{j , i} \vert < t\end{matrix} \}$$

Where and x_{j,i} are x′_{j,i} the noise and denoise coefficients of the wavelet at a j^{th} decomposing level and i location. Finally, the shrunk coefficients are first rescaled (p) and then inversely transform to the original domain which is the denoised signal.

To check the performance of the filtered data signal to noise ratio (SNR) and its root mean square error (RMSE) can be determined as below:

$$RMSE = \sqrt{\frac{1}{N} {\sum }_{n = 1}^{N} {[ x ( n ) - {x}^{′} ( n ) ]}^{2}} \\ SNR = 10 {log}_{10} \{ \frac{{\sum }_{n = 1}^{N} {[ x ( n ) ]}^{2}}{{\sum }_{n = 1}^{N} {[ x ( n ) - {x}^{′} ( n ) ]}^{2}} \}$$

### EXPERIMENTAL RESULTS

The section below includes experimental data from two sets of experiments. In the first, a single magnetic sensor was used. In the second set of data, a quad sensor (i.e. a sensing arrangement including four magnetic sensors, such as the one shown in FIG. 10) was utilized.

A. Single Sensor Configuration

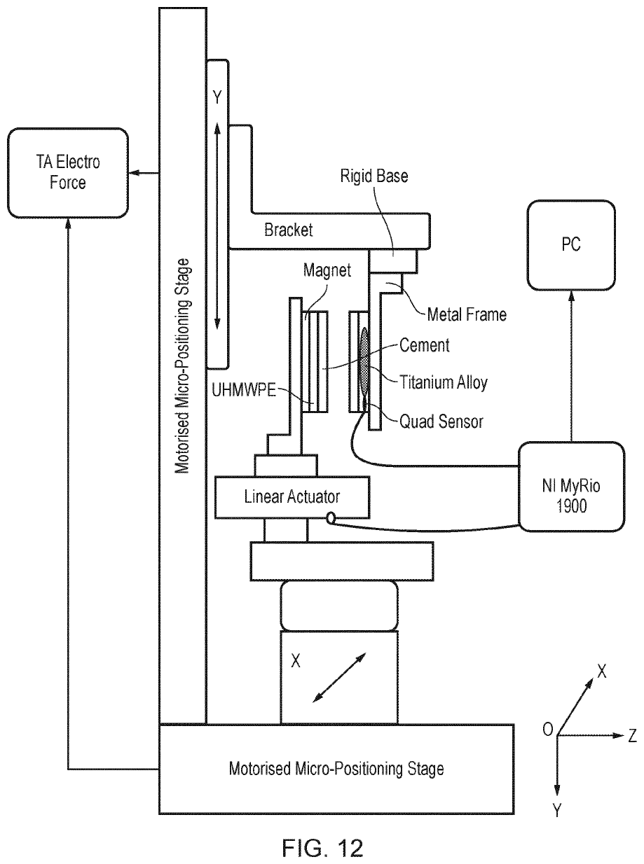

To evaluate the performance of the sensor and to obtain the correlation between the magnetic field and displacement, a mechanical testing system, shown in FIG. 12, (TA, Electro Force 3300, Boston, USA) was used to provide input migration of the implant via its two motorized stages i.e. linear and rotational with the resolution of 0.5 μm linearly, 0.01 degree angularly and 0.01 Hz frequency. To provide a repeatable simulation of implant migration, the inventors designed and fabricated an adjustable fixture/bracket for holding the sensor and magnet embedded in a cement restrictor. The fixture was attached with the Electro-force machine. One of the motorized motors moved the sensor bracket linearly in the y axis while the other can move the magnet bracket rotationally in the x/z plane. In order to move the magnet bracket in z-axis a linear actuator was attached with the resolution of 1 mm and this was used to adjust the distance between the sensor and magnet. The Electro-Force machine was programmed to move in the y axis quasi-statically (3 minute intervals) at amplitudes of 0.3 to 4 mm using square waveforms and dynamically using sine waveform at 0.1 Hz. Similarly the machine was programmed to move in the x/z plane (rotation) quasi-statically. The z direction was controlled via the linear actuator, which was programmed using an external DAQ card (NI LabView) the DAQ card was also used to communicate and acquire data with magnetic sensor and to record data into a measurement file.

Sensor Calibration

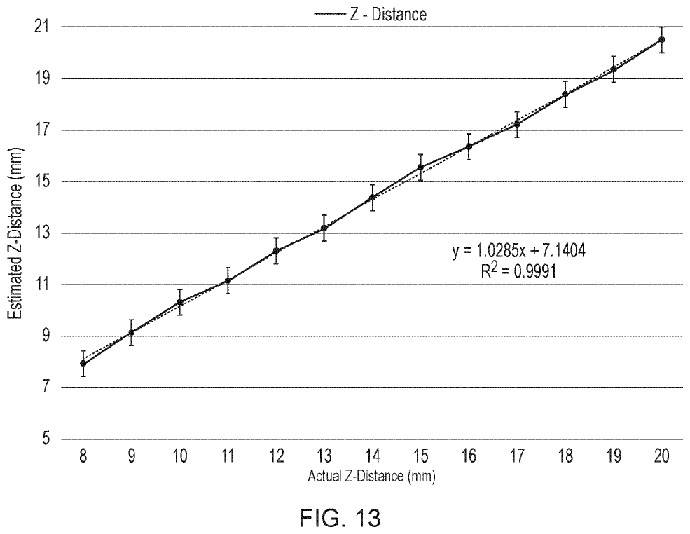

The sensor was first calibrated in the z-axis only in order to estimate the distance between the sensor and magnet. The permanent magnet was placed perpendicular to the z-axis of the sensor (Note that North Pole was facing towards the sensor, if poles changes the magnetic field sign change from positive to negative) and was moved linearly with the range of 18 mm at a step size of 1 mm. The resulted data set was processed with the algorithm as shown in FIG. 8 to determine the z-distances. FIG. 13 shows the comparison of the estimated distance with actual distance. The result showed how well the algorithm fitted the actual value with R² close to 0.9991.

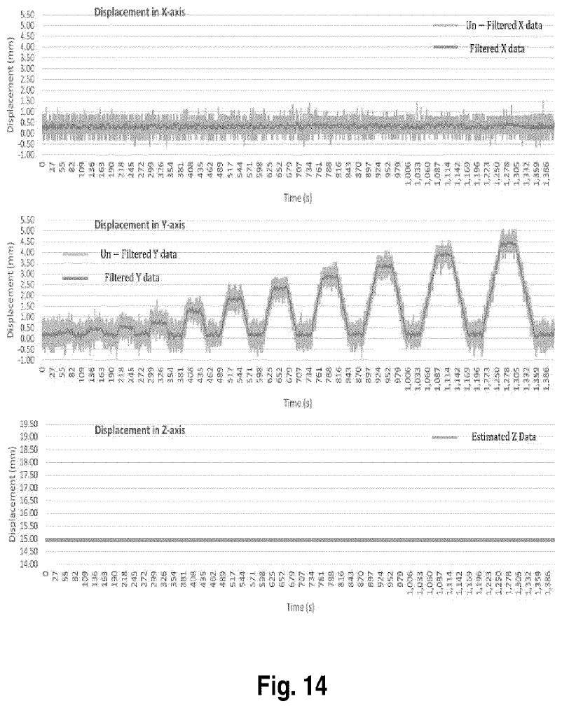

After estimating the distance between sensor and magnet, a similar set of experiments were conducted to analyse how the system detected the displacement of the sensor/magnet if they moved in a non-z direction. To evaluate this, the sensor was first moved linearly in y-axis ranging from 0.1 mm to 4 mm with the step size of 0.5 mm (at z=15 mm). It was and also moved angularly around the y-axis ranging from 0 to 4.0 degrees. FIG. 14 shows that the system was able to detect the displacement of magnet in the y-axis with no change in the x-axis showing a resolution of up to 0.3 mm. Displacement detection at different movement are provided in Table 1 for both filtered and unfiltered signals. The filtered version shows a considerably low standard deviation at 0.3 mm movement of 0.079 compared to the unfiltered standard deviation of 0.39. Similar results were seen during x-axis linear movement where the y-distance remained constant.

TABLE 1
Real and estimated values of movement in the y-direction.

| Actual | Estimated Movement | Estimated Movement |
|---|---|---|
| movement (mm) | without filter (mm) | with filter (mm) |
| 0.3 | 0.321 ± 0.390 | 0.328 ± 0.079 |
| 0.5 | 0.527 ± 0.393 | 0.530 ± 0.075 |
| 1.0 | 1.078 ± 0.386 | 1.082 ± 0.089 |
| 1.5 | 1.627 ± 0.384 | 1.632 ± 0.074 |
| 2.0 | 2.126 ± 0.372 | 2.133 ± 0.079 |
| 2.5 | 2.660 ± 0.371 | 2.665 ± 0.080 |
| 3.0 | 3.169 ± 0.380 | 3.173 ± 0.079 |
| 3.5 | 3.701 ± 0.371 | 3.705 ± 0.078 |
| 4.0 | 4.191 ± 0.369 | 4.195 ± 0.085 |

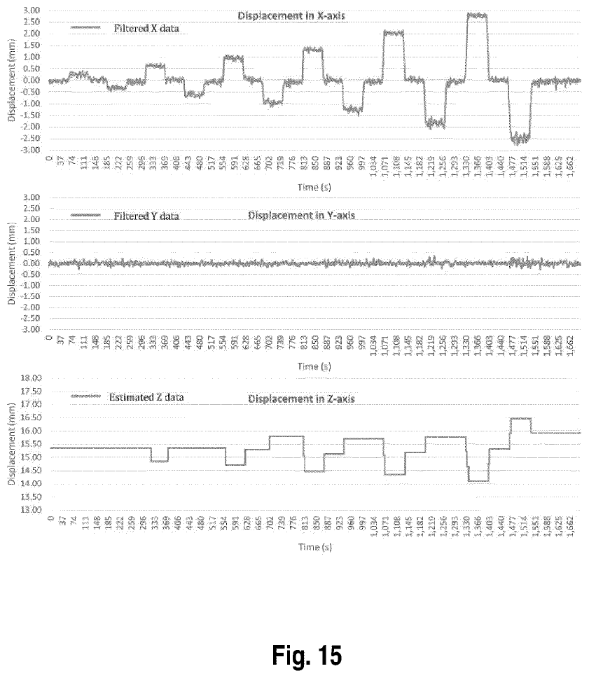

FIG. 15 shows the angular movement of the magnet around the sensor. The system was able to detect angular displacement until 3.0 degrees (approximately 2 mm). Also, from FIG. 15 it can be observed that up to 1 degree rotation (1.2 mm angular displacement) there is no change in the y movement but beyond 1 degree the y displacement start to increase. Also, it was observe that beyond 3.0 mm the sensor was able to detect the magnetic field but it introduced error in the tracking algorithm this error was due to the tilting effecting of the sensor.

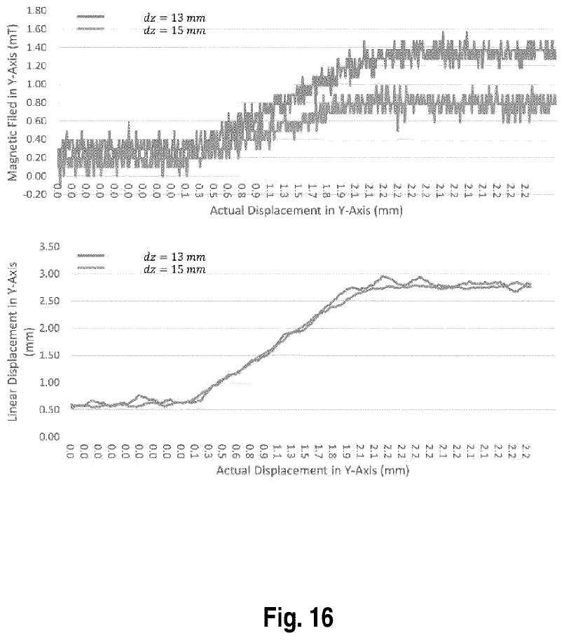

The graphs in FIG. 16 show the comparison of magnetic field (B_{y}) with the calibrated sensor output during the y-axis displacement at different z values. It was observed that during the y-axis displacement at different z-distances the magnetic field (B_{y}) changes, demonstrating a strong cross-talk effect, whereas the output from the calibrated sensor demonstrated a strong correlation with the actual y-distance across different z values. In other words, the undesirable cross-talk effect can be eliminated using the system of the present invention.

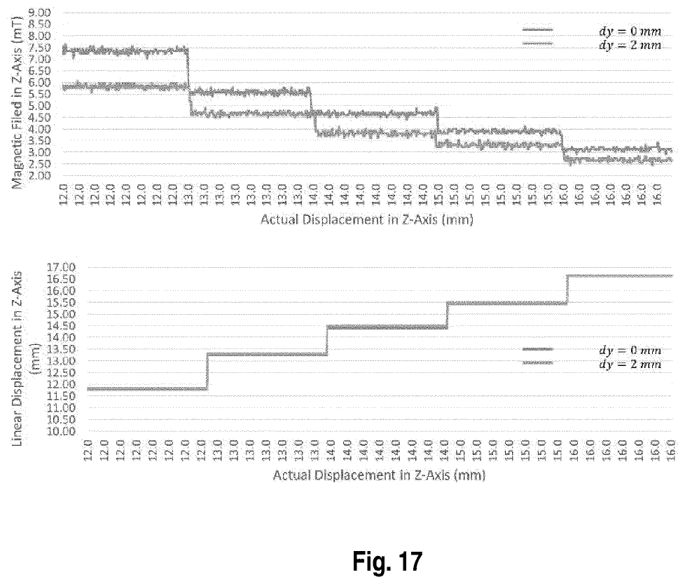

Also, the graphs in FIG. 17 show that during z-axis displacement at different y-distances, similar effects were observed with strong cross-talk in magnetic field (Bz) which is eliminated when using the calibrated sensor.

Sensor Performance for Different Movements and Materials

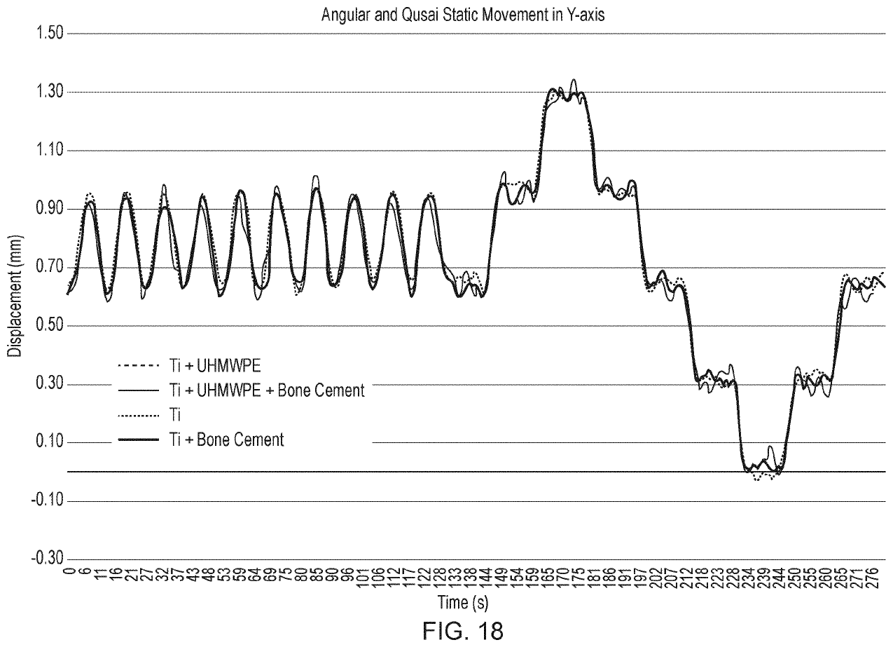

The calibration steps described above, and whose results were shown in FIGS. 13 to 17, were performed without introducing any material between the sensor and magnet. To further investigate the performance of the sensor, the system was tested with the biomaterials that resembles the environment, that are present in the elbow prosthesis, namely, PMMA cement, UHMWPE and Titanium alloy (Ti-6Al-4V). FIG. 18 shows that these materials have negligible effect on the measuring system, as do the results in Table 2.

TABLE 2
Mean and standard deviations of static and dynamic
distances for a range of different materials.

| | Static Movement | Dynamic Movement |
|---|---|---|
| Materials | (0.3 mm) | (0.3 mm) |
| No Material | 0.3091 ±0.023 | 0.3133 ± 0.1012 |
| Titanium | 0.3191 ± 0.023 | 0.3231 ± 0.1040 |
| Titanium and Bone cement | 0.3085 ± 0.033 | 0.3305 ± 0.1212 |
| Titanium and UHMWPE | 0.3043 ± 0.028 | 0.3323 ± 0.11175 |
| Titanium, Bone cement, | 0.3085 ± 0.033 | 0.3317 ± 0.1221 |
| and UHMWPE | | |

Sensitivity

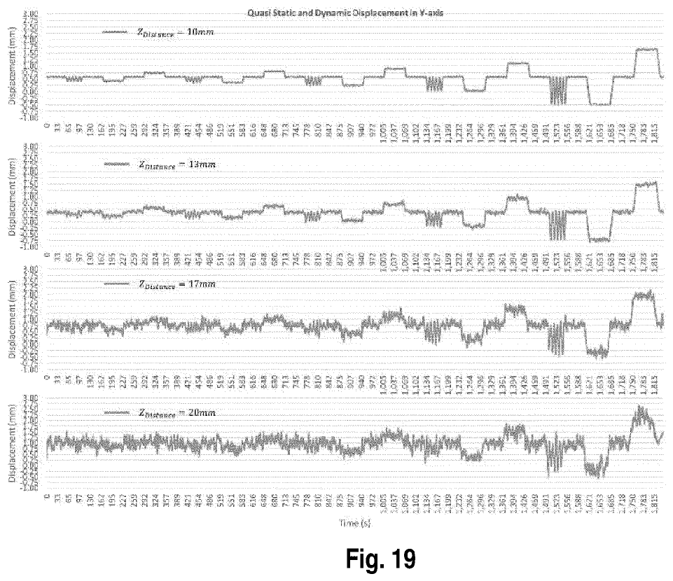

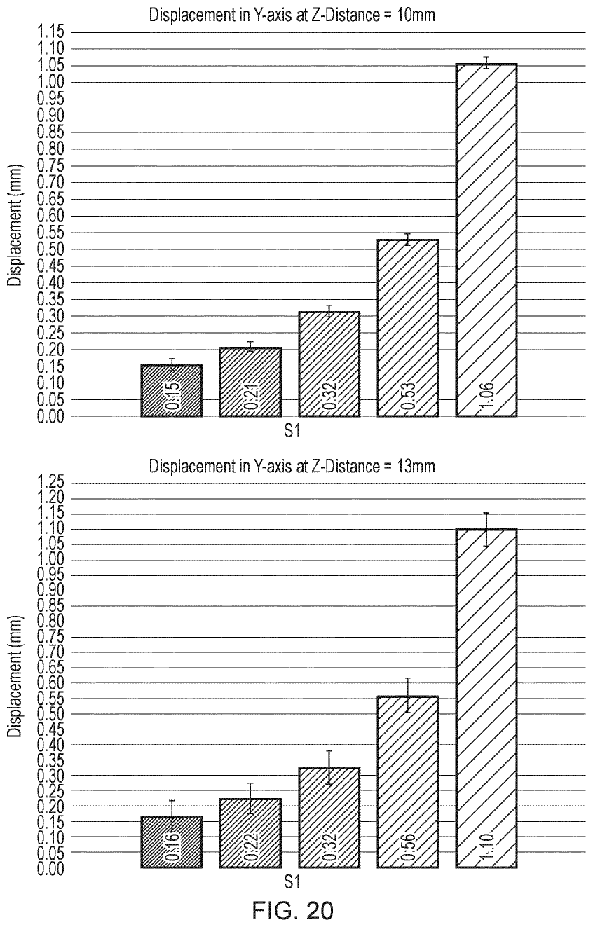

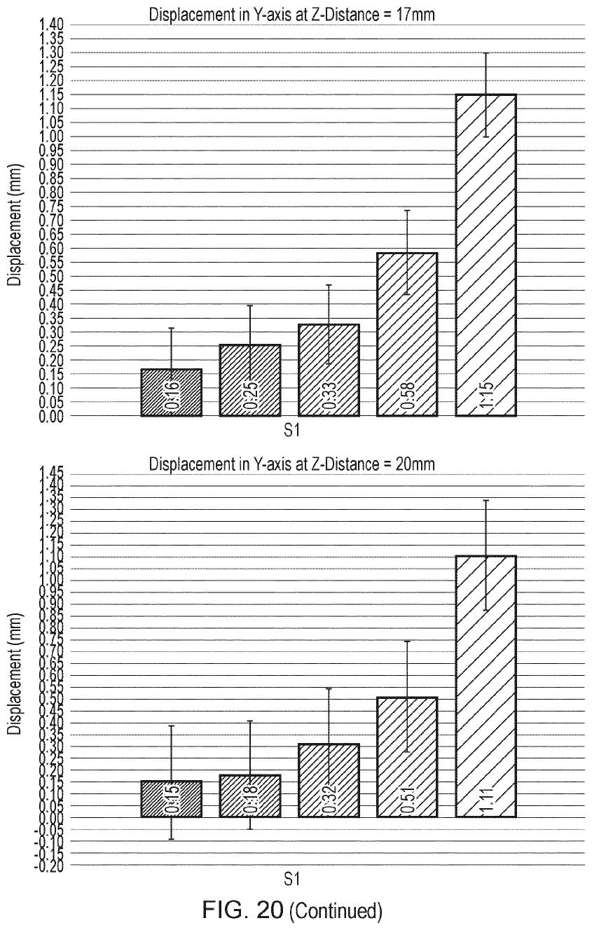

In the literature, there is no specific information about the minimum distance between the humeral tip and the cement restrictor in total elbow arthroplasty. However, in shoulder arthroplasty the minimum distance between cement restrictor and humeral component is around 10 mm. In order to check the sensitivity of the system of the present invention, the inventors placed the permanent magnet in the cement restrictor at a distance of greater than 10 mm from the magnetic sensor and showed the system performs under static and dynamic linear movement in they direction ranging from 0.15 mm to 1 mm. FIGS. 19 and 20 show that as the distance between sensor and magnet is increased the resolution of the system decreases and more noise content are introduce to the signal. At z=15 mm, the system was able accurately to detect static and dynamic movement. Beyond this distance 0.5 mm static movement was detected but with higher noise content. Specifically, FIG. 20 shows the sensitivity of the single sensor configuration with respect to various z-distance values when detecting linear movement in the y-direct. Also, the bars in the graph represent different implant movement e.g. the left-most bar shows the results when the implant is moved 0.15 mm, the next bar 0.20 mm, the next bar 0.30 mm, the next bar 0.50 mm and the right-most bar 1.00 mm.

The results show that, ideally, the distance between sensor and magnet for static and dynamic displacement should be below 16 mm.

Linear and Angular Movement

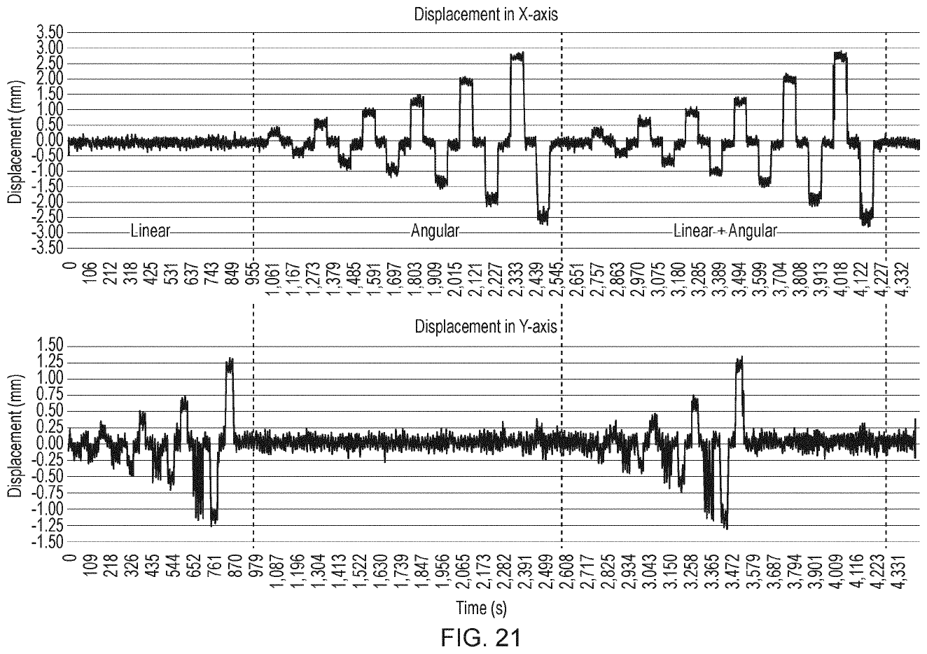

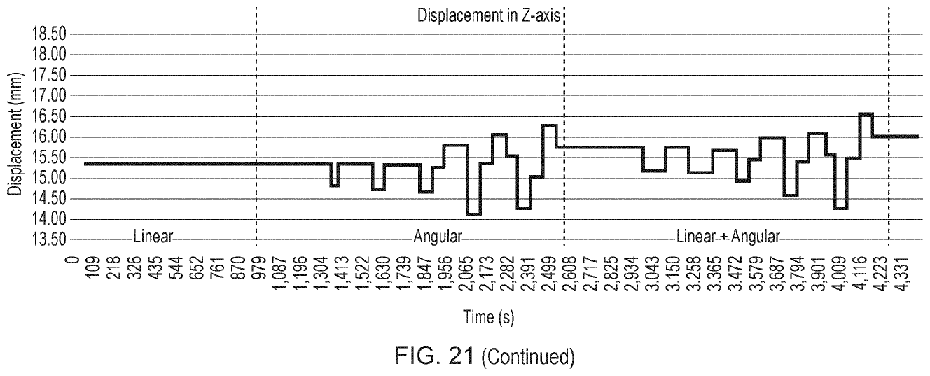

FIG. 21 shows that the system was able to detect linear and angular displacement simultaneously. As describe previously, there was a small increment in the y axis movement when angular displacement exceed 1.2 mm (1 degree).

B. Quad Sensor Configuration

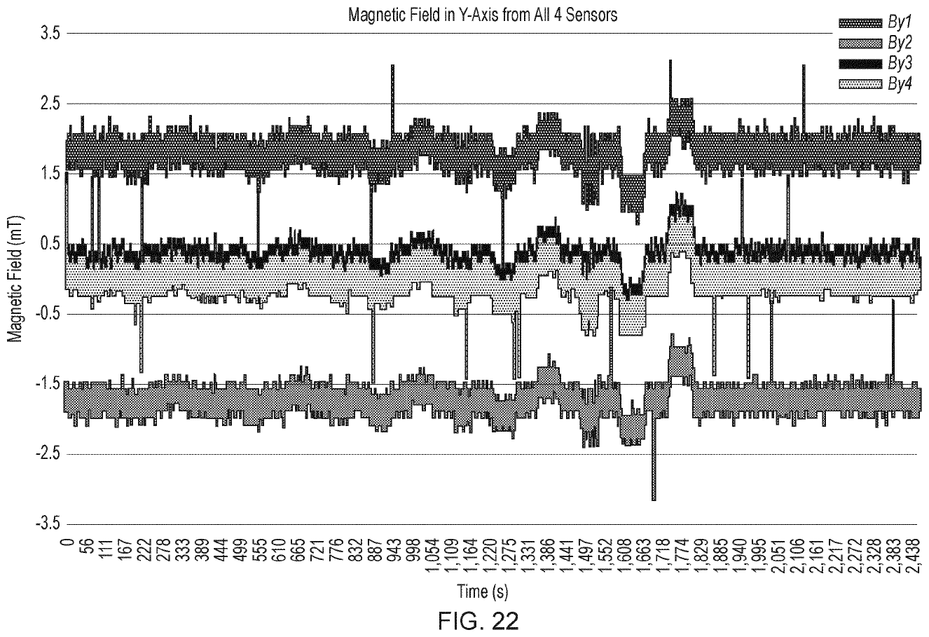

A similar experimental setup was used to analyse the quad sensor arrangement, as for the single sensor arrangement. First, the effect of the magnetic field on the quad sensor configuration was analysed. As shown in FIG. 10, the sensors are very closely placed to each other. This means that if there is any type of magnetic variation all the sensors should read the same effect. FIG. 22 shows the variation in the y-component of the magnetic field strength, when the permanent magnet was randomly moved.

It can be seen that the y-components of the magnetic field strength of all four sensors change in unison as the permanent magnet is moved in the y-direction, but with different magnitude because of their position. The magnetic field of sensors 3 and 4 were almost the same because their y-distance values are the same. In contrast, because sensors 1 and 2 are 6 mm apart from each other in the y-direction, they have a magnetic field magnitude difference. Similarly, when the magnet was moved in x-axis all the sensors showed the same magnetic field variation response but with different magnitude.

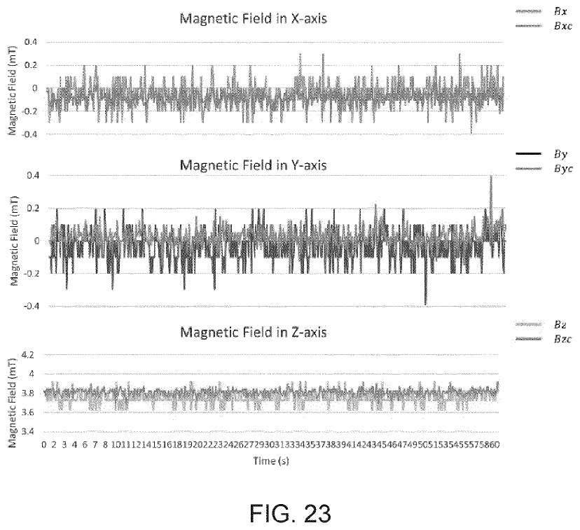

As shown in FIG. 23, the magnetic field strengths measured by the quad sensor configuration show a reduction in the noise content as compared with the single sensor configuration. This reduction was observed even without the application of any kind of filtering technique to it. FIG. 23 shows the raw magnetic field strength data measured by the single sensor configuration and quad sensor configuration, for each of the x-, y-, and z-components of the magnetic field strength. This reduction in noise which is exhibited in the data from the the quad sensor configuration arises due to the cancellation of external magnetic fields: the sensors each measure the same amount of external magnetic field so by combining them external magnetic field variation is cancelled.

Z-Distance Estimation

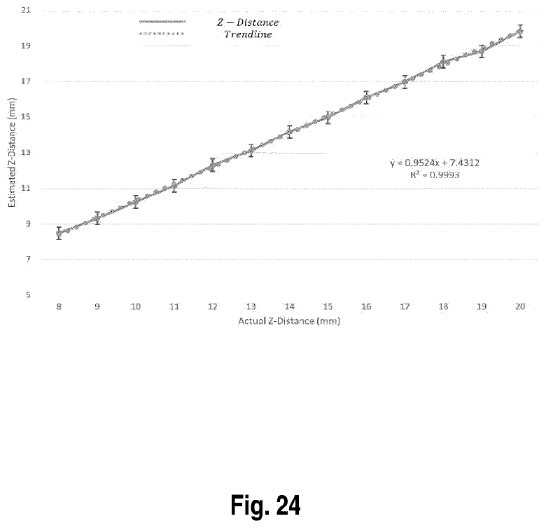

As described in the previous section the quad sensor needs to be calibrated in order to determine the localization of the magnet. The first step of the detection algorithm is to determine the z-distance between the sensor and magnet. In order to check the effect of the quad sensor in estimating the z-distance, the permanent magnet enclosed in cement restrictor was placed perpendicularly, pointing towards to the quad sensor that was hermetically sealed in 2 mm thick titanium alloy. By using the linear actuator the distance between the quad sensor and magnet was increased with a step size of 1 mm. FIG. 24 shows that the estimated distance result from the quad sensor configuration matches well with the actual distance with a R² values of 0.9993.

Linear Movement in y-Direction

As discussed in the calibration section, displacement detection in the x and y-axis can be determined by using the equations given earlier in this application. In order to analyse the performance of the quad sensor in detecting movement in the x- and y-directions. The sensor was first moved linearly in the y-direction ranging from 0.15 to 4.0 mm with a step size of 0.5 mm, keeping the x-distance constant and with a z-Distance of 15 mm. Compared with the single sensor configuration, the noise content in the quad sensor is low as previously described. However, additional filtering is carried out here, in order to smooth the data. Table 3 below shows the selected type of DWT filter along with its parameters. The selection of mother wavelet and decomposition was based on the signal reconstruction and SNR value.

TABLE 3
Selected parameters for DWT

| | 1^{st} Step | | 2^{nd} Step | | 3^{rd} Step |
|---|---|---|---|---|---|
| | Mother | Decomposition | Threshold | Threshold | Threshold |
| Parameters | Wavelet | level | Function | Selection | rescaling |
| Selected | Sym 6 | 6 | SURE | Hard | one |
| Parameter | | | | | |

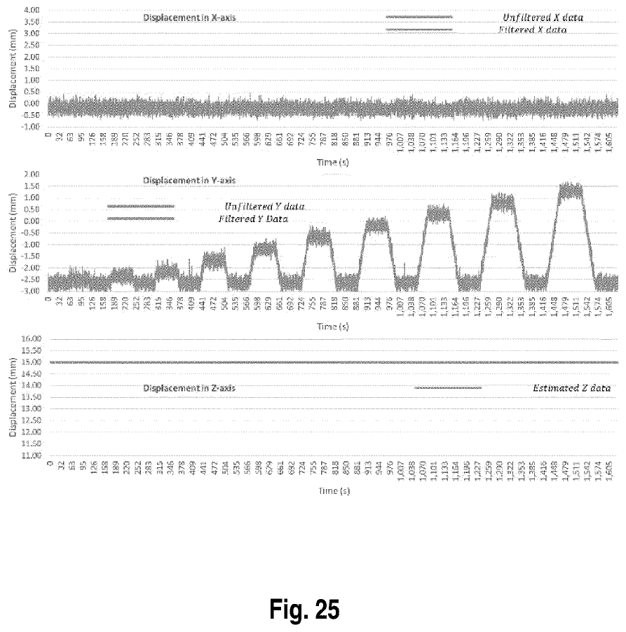

FIG. 25 shows both filtered and unfiltered x-, y-distances along with the z-Distance estimation value. The result from the quad sensor configuration showed a well-matched linear displacement detection with a resolution of 0.15 mm.

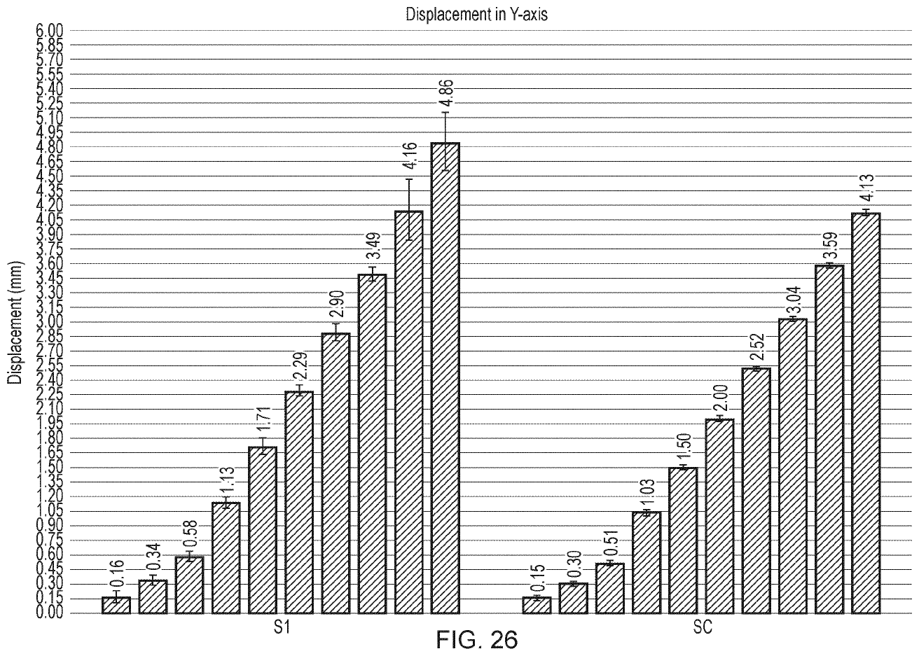

FIG. 26 shows a comparison of a single sensor configuration with a quad sensor configuration, keeping the z-distance 15 mm. Single sensor configuration has a resolution of 0.3 mm while the quad sensor has a 0.15 mm. In the quad sensor configuration, the standard deviation error and mean error remained minimal as compared to single sensor configuration, the standard deviation error and mean error of which increased with the increase of displacement. The bars in the graph are used to differentiate the different linear movement in y-axis. Starting from the left: 0.15 mm, 0.30 mm, 0.50 mm, 1.0 mm, 1.5 mm, 2.0 mm, 2.5 mm, 3.0 mm, 3.5 mm, 4.0 m.

TABLE 4
Comparison of RMSE and SNR for single and quad sensor configurations.

| Displacement | Single Sensor Configuration | | Quad Sensor Configuration | |
|---|---|---|---|---|
| (mm) | RMSE | SNR | RMSR | SNR |
| 0.15 | 0.063 | 33.27 | 0.031 | 39.34 |
| 0.30 | 0.051 | 41.14 | 0.032 | 45.30 |
| 0.50 | 0.066 | 43.36 | 0.021 | 53.51 |
| 1.00 | 0.137 | 43.03 | 0.026 | 57.59 |
| 1.50 | 0.157 | 45.40 | 0.035 | 58.47 |
| 2.00 | 0.222 | 44.87 | 0.051 | 57.72 |
| 2.50 | 0.288 | 44.55 | 0.047 | 60.31 |
| 3.00 | 0.355 | 44.32 | 0.033 | 64.92 |
| 3.50 | 0.390 | 44.64 | 0.026 | 68.41 |
| 4.00 | 0.479 | 44.21 | 0.020 | 71.83 |

Table 4 describes the RMSE and SNR between both configurations. It was observed that in the single sensor configuration the maximum SNR value was 45 dB at 1.50 mm displacement and with increase in distance the RMSE increased. While, in quad senor configuration with increase in distance the SNR value increased and this configuration showed a maximum RMSE of 0.051.

Angular Movement in x-Direction

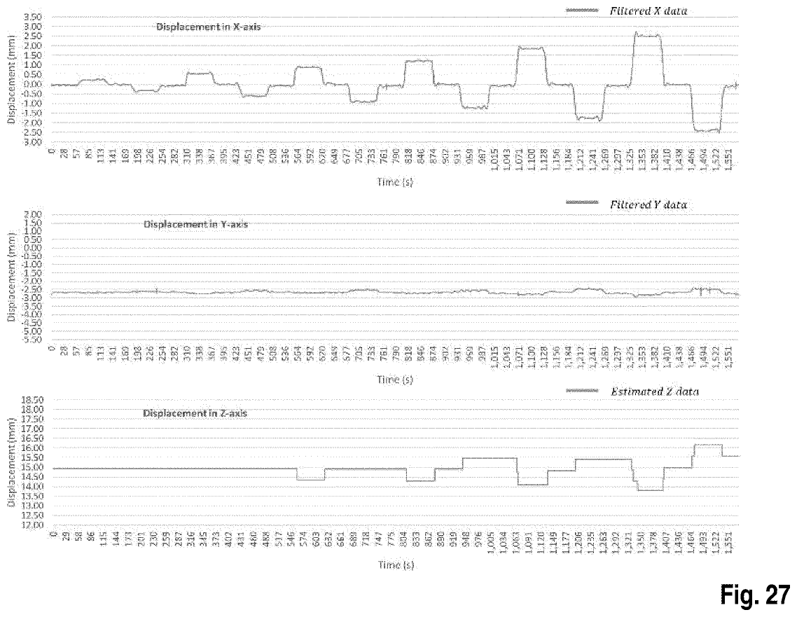

FIG. 27 shows the results obtained in the quad sensor configuration, when the magnet was moved angularly about the x-axis ranging from 0.5 degrees to 4.0 degrees with a step size of 0.5 degrees keeping the position with respect to the y-axis constant and at Z-Distance of 15 mm. Up to 1 degree of movement (approximately 0.60 mm) the z-distance remains same, but beyond 1 degree there was a change in the z-distance value. This change in value was only observed when the magnet was angular moved in a negative direction up to 1.5 degrees. After 2 degrees the same change in the z-distance was observed in both negative and positive axis. Also, there was a slight change in the y-distance when the magnet was angularly moved beyond 2 degrees.

The change in z-distance arises because as the magnet angularly moves it changes the distance between the sensor and magnet, which our algorithm was able to detect. Also, the in z-distance can differentiate between linear and angular movement. The change in the y-distance is due to the orientation of the magnet.

FIG. 27 shows a comparison of a single sensor configuration with a quad sensor configuration during angular motion. As described previously, the main drawback of single sensor configuration was the effect of tilt. Due to this effect, the single sensor configuration was only able to detect movement within 3 degrees depending upon its orientation position. In the quad sensor configuration, the tilting effect is resolved. This was achieved by introducing specific sensor selection criteria.

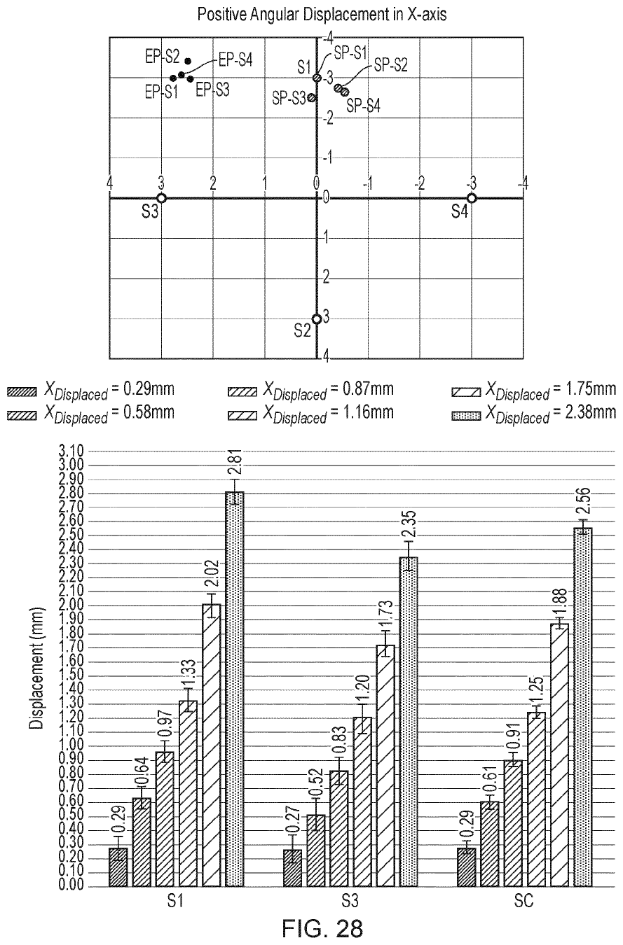

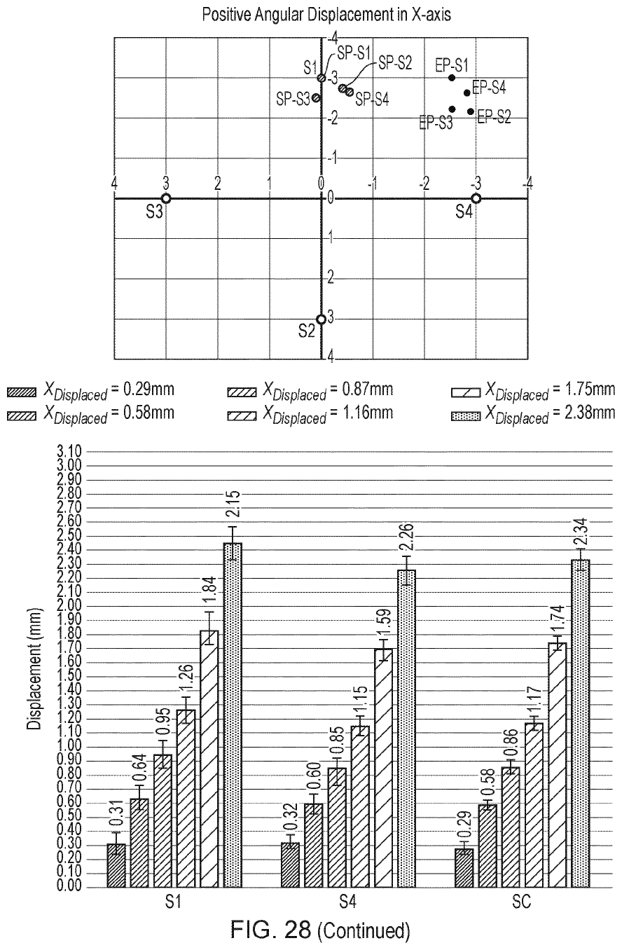

FIG. 28 shows the position of the magnet observed by all four sensors. In the drawing, S1, S2, S3, S4 show the position of the sensors, while SP-S1 to SP-S4 show the starting position of the magnet when there was no angular movement and EP-S1 to EP-S4 show the end position of the magnet when it was angularly moved 4 degrees (approximately 2.3 mm) in both positive and negative direction.

This shows that the quad sensor configuration of the present invention clearly specifies the position of the magnet. During the angular movement experiment, the starting position of the magnet was at the centre of sensor S1, then moved 4 degrees in both the positive and negative directions. As discussed, the single sensor can detect angular displacement up to 3 degrees depending upon the orientation. In FIG. 28 during the positive x-direction displacement, S1 was only able to measure the till 1.5 degrees beyond that the mean error and standard deviation error started to increase. Similarly, the quad configuration also showed the same response. By introducing the sensor selection criteria this tilting effect was compensated.

The sensor selection criteria depend upon the localization of the magnet. It can be observed that during positive x-direction displacement, the magnet is displaced between sensor S1 and S3. Therefore eliminating S2 and S4 value from the quad configuration gives a well-matched result. Similarly, during the negative x-direction displacement the single sensor and quad sensor configurations were able to detect the movement with minimum error, but as explained previously as the magnet lies between sensor S1 and S4. Therefore, by combining the values of these sensors and eliminating sensors S2 and S3 gives better, well-matched results.

Cross-Talk

The magnetic field density of a permanent cylindrical magnet displays two features: non-linearity and cross talk. Due to these, the magnetic field does not vary linearly with spatial location, and movement in a single can direction (e.g. the x-direction) can lead to a change in all three of the components of the magnetic field strength, which is experienced by e.g. the magnetic sensor. These features give rise to a correlation between displacement and magnetic field which is not-insignificant and challenging to resolve.

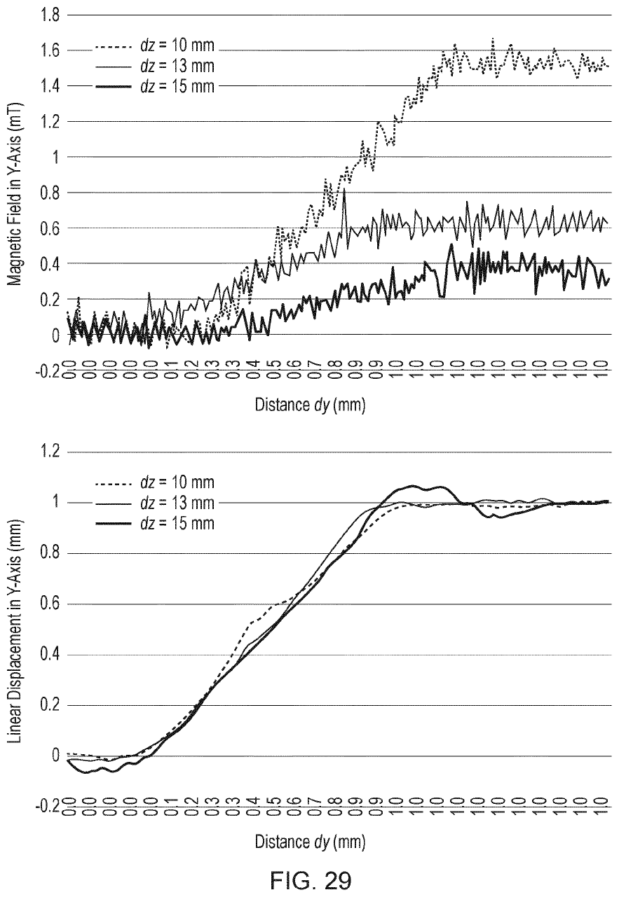

FIG. 29 shows the y-component of the magnetic field strength, as measured by the quad sensor, when the magnet was moved to 1 mm with a step size of 0.1 mm at different z-distance positions. FIG. 29 (LHS) shows that without calibration the y-component of the magnetic field strength, with varying z-distances shows a strong cross talk effect, along with non-linearity.

FIG. 29 (RHS) shows that the calibrated output from the quad sensor shows a close agreement with the actual displacement, along with elimination of the cross talk effect.

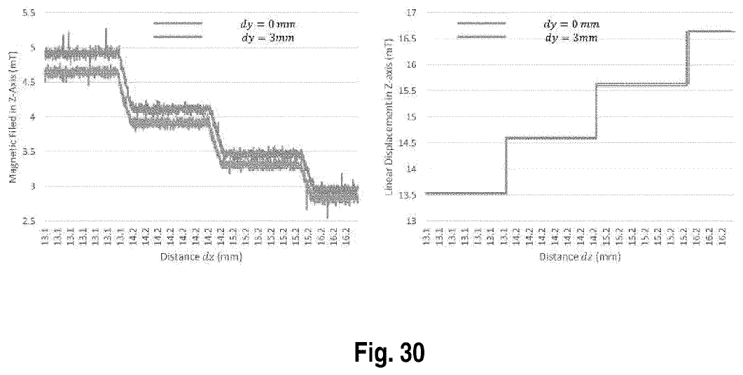

A similar set of experiments was conducted to analyse the effect of the correlation of magnetic field with respect to displacement by moving the magnet in the z-direction to 4 mm with the step size of 1 mm at different y-distances. FIG. 30 (LHS) demonstrates the same effect of strong cross-talk and non-linearity as the magnet was moved in z-axis at different y distance value, and FIG. 30 (RHS) shows the calibrated sensor output, clearly demonstrating elimination of the cross talk effect.

Resolution

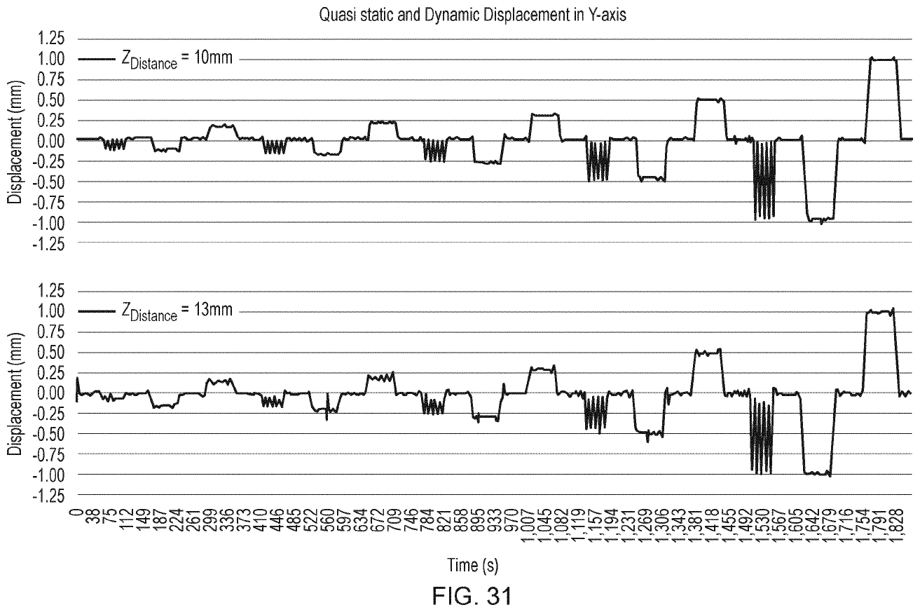

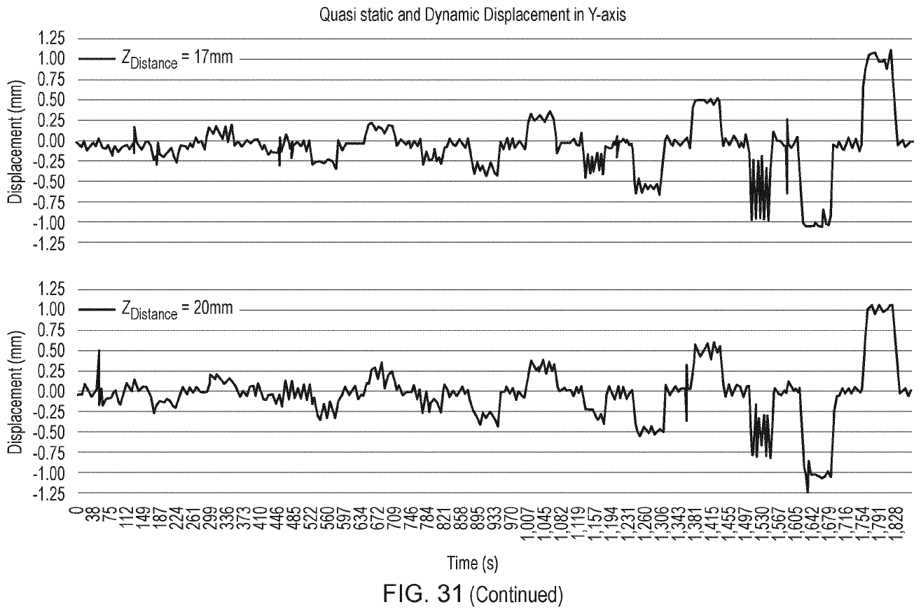

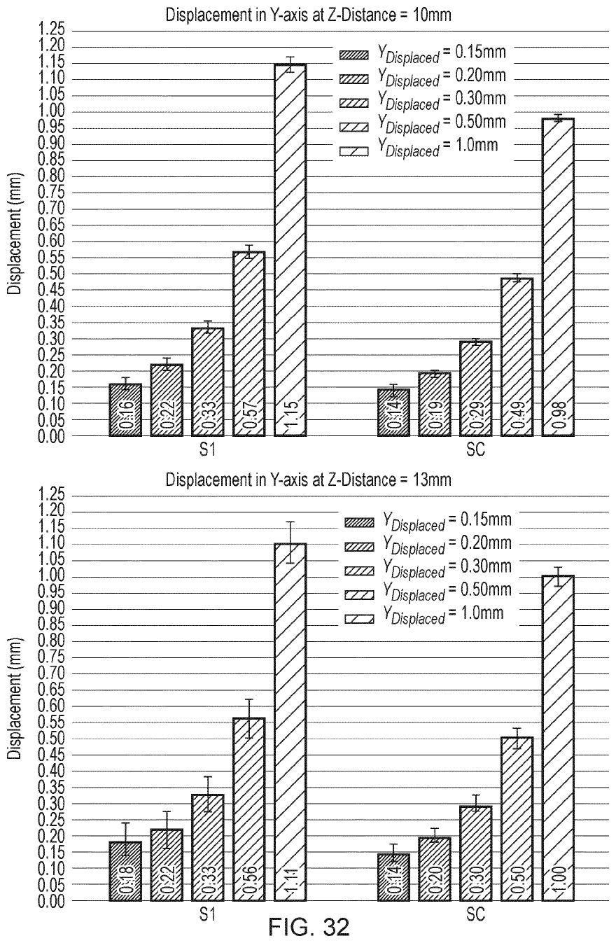

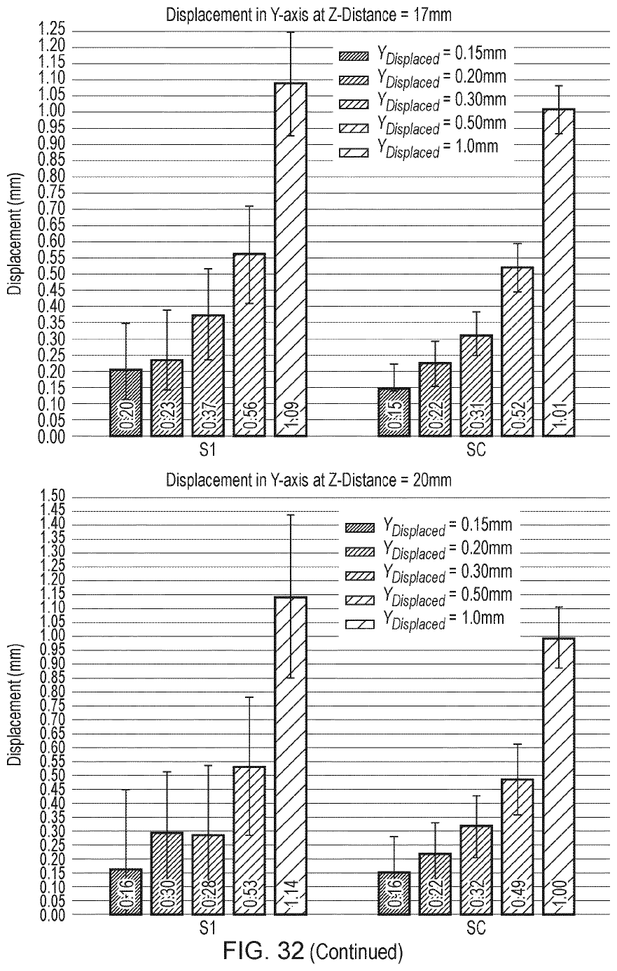

To analyse the sensitivity of the quad sensor configuration in static and dynamic movement. 4 sets of experiments were conducted by moving the magnet quasi statically and dynamically in the y-direction to 1 mm with a set of 0.15, 0.20, 0.30, 0.50 and 1.00 mm at different z-distances. The selection of z-distances was based on the teaching of the literature, that the minimum distance between the humeral tip and cement restrictor is 10 mm. So, the starting z-distance was selected to be 10 mm and the end z-distance 20 mm. The results of these experiments are shown in FIGS. 30 and 31.

1. A system for tracking relative displacement between a first element and a second element in a structure, the system including:
a permanent magnet fixed relative to the first element;
a magnetic sensor fixed relative to the second element, the magnetic sensor configured to measure a magnetic field strength; and
a processor configured to determine the relative displacement between the first element and the second element, based on the magnetic field strength measured by the magnetic sensor.

2. The system according to claim 1, wherein:
the magnetic sensor is configured to generate magnetic field strength data based on the measured magnetic field strength, and to transmit the generated magnetic field strength data to the processor.

3. The system according to claim 2, wherein:
the magnetic sensor is configured to measure the magnetic field strength, and thereby to generate the magnetic field strength data at predetermined intervals.

4. The system according to claim 1, wherein:
the magnetic sensor includes three one-dimensional sub-sensors arranged mutually orthogonally, each of the three one-dimensional sensors configured to measure a magnitude of a respective one of the three orthogonal components of the magnetic field at the magnetic sensor; and
the direction of the component of the magnetic field at the magnetic sensor of which a given sub-sensor is configured to measure or determine the magnitude is referred to the “orientation” of that sub-sensor.

5. The system according to claim 4, wherein:
the orientations of the three sub-sensors correspond to the axes of the magnetic sensor.

6. The system according to claim 5, wherein:
the permanent magnet is a cylindrical magnet, the z-axis of which is initially aligned with and is parallel to the z-axis of the magnetic sensor.

7. The system according to claim 4, wherein:
the magnetic sensor is configured to determine the value of the magnitude of the component of the magnetic field strength of the permanent magnet which is along the z-axis of the permanent magnet; and
the processor is configured to determine the z-distance between the permanent magnet and the magnetic sensor using the value of the magnitude of the component of the magnetic field strength of the permanent magnet which is along the z-axis of the permanent magnet.

8. The system according to claim 7, wherein:
the processor is configured to determine the x-distance based on at least the z-distance, and the values of the magnitudes of the x- and z-components of the magnetic field strength at the magnetic sensor, measured by the magnetic sensor; and/or
the processor is configured to determine the y-distance based on at least the z-distance, and the values of the magnitudes of the y- and z-components of the magnetic field strength at the magnetic sensor, measured by the magnetic sensor.

9. The system according to claim 7, wherein:
the processor is configured periodically to update the value of the z-distance used to calculate the x-distance and the y-distance.

10. The system according to claim 9, wherein:
after a predetermined amount of time or a predetermined number of measurements have been taken by the magnetic sensor, the process is configured to:
calculate a new z-distance;
compare the new z-distance with the old z-distance; and
if the new z-distance differs from the old-distance by more than or equal to a predetermined distance threshold, the processor is configured to adopt the new z-distance; and
if the new z-distance differs from the old z-distance by less than the predetermined difference threshold, the processor is configured to reject the new z-distance, and to continue calculating the x-distance and the y-distance using the old z-distance.

11. The system according to claim 1, wherein:
the system includes a plurality of magnetic sensors.

12. The system according to claim 11, wherein:
the plurality of magnetic sensors includes a first pair of sensors consisting of a first sensor and a second sensor, and a second pair of sensors consisting of a third sensor and a fourth sensor;
the plurality of magnetic sensors are arranged in a cross formation, in which the first pair of sensors are spaced from each other in a first direction, and the second pair of sensors are spaced from each other in a second direction which is perpendicular or substantially perpendicular to the first direction.

13. The system according to claim 12, wherein:
the plurality of sensors are arranged in the x-y plane;
the value of the magnitude of the z-component of the magnetic field strength at the plurality of sensors is calculated by taking an average of the value of the magnitude of the z-component of the magnetic field strength measured by each of the plurality of the magnetic sensors.

14. The system according to claim 13, wherein:
the values of the magnitudes of the x-component and/or y-component of the magnetic field strength at the plurality of sensors are calculated as a linear superposition of the values of the magnitudes of the x-component and/or y-component of the magnetic field strength measured by each of the plurality of magnetic sensors.

15. The system according to claim 1, wherein:
the processor is configured to apply Savitzky-Golay filter or a modified Savitzky-Golay filter to the magnetic field strength data or the relative displacement data in order to smooth it and to remove high frequency noise.

16. The system according to claim 1, wherein:
the processor is configured to remove noise from the magnetic field strength data or relative displacement data by using a discrete wavelet transform (DWT) denoising technique.

17. The system according to claim 16, wherein:
the DWT denoising technique includes the following steps:
transforming the data into the wavelet domain;
defining a decomposition level;
reducing selected components of the coefficients of the wavelet transform;
rescaling the reduced coefficients; and
inversely transforming the wavelet transform, to give the denoised signal.

18. The system according to claim 1, wherein:
the first element includes an orthopaedic prosthesis; and
the second element includes a component which is fixable to a bone, such that the permanent magnet is fixed relative to the bone.

19. The system according to claim 18, wherein:
the system further includes:
bone cement filling a space between the prosthesis and bone; and
a cement restrictor configures to prevent the bone cement from diffusing into the bone.

20. The system according to claim 19, wherein:
the orthopaedic prosthesis is an artificial elbow joint.

21. The system according to claim 18, wherein:
the magnetic sensor is attached to or embedded into the orthopaedic prosthesis.

22. The system according to claim 18, wherein:
the magnet is either: fixable to the bone, fixed to the cement restrictor, or embedded within the bone cement.

23. A method of tracking relative displacement between a first element and a second element in a structure, the structure including a magnet fixed to the first element and a magnetic sensor fixed to the second element, and the method including the steps of:
measuring, using the magnetic sensor, a magnetic field strength; and
determining the relative displacement between the relative displacement between the first element and the second element, based on the magnetic field strength measured by the magnetic sensor.
